(19) United States
(12) Patent Application Publication
(10) Pub. No.: US 2011/0047646 A1
(43) Pub. Date: Feb. 24, 2011

(54) Armyworm Insect Resistance Management in Transgenic Plants

(76) Inventor: Juan Ferre Manzanero, Valencia (ES)

Correspondence Address: BAYER CROPSCIENCE LP, Patent Department, 2 T .W. ALEXANDER DRIVE, RESEARCH TRIANGLE PARK, NC, 27709, US

(21) Appl. No.: 12/990,327

(22) PCT Filed: Apr. 30, 2009
(86) PCT No.: PCT/EP09/03130

§ 371 Date: Oct. 29, 2010

Related U.S. Application Data
(60) Provisional application No. 61/126,083, filed on May 1, 2008.
(60) Provisional application No. 61/195,019, filed on Oct. 2, 2008.

(51) Int. Cl. A01H 5/00; A01N 37/18; A01P 7/04; C12N 15/82; A01G 1/00; A01C 7/00

(52) U.S. Cl. 800/279; 514/4.5; 800/302; 47/58.1 R; 111/100

(57) ABSTRACT

This invention relates to a process for preventing or delaying the development of resistance in populations of Spodoptera frugiperda to transgenic plants expressing a Cry1A and/or a Cry1F protein, comprising providing such plants also with a gene expressing a VIP3 protein, as well as to related uses and methods, such as methods for the production of transgenic plants comprising two different insecticidal proteins that show no competition for binding to the binding sites in the midgut brush border of Spodoptera frugiperda larvae.

### FIELD OF THE INVENTION

The present invention relates to the field of plant pest control, particularly insect control. This invention relates to the use of transgenic plant cells and plants in an insect resistance management process, wherein the genomes of said cells and plants (or more typically, predecessor plant cells or plants) have been provided with at least two genes, each encoding a different protein insecticidal to Spodoptera frugiperda, which proteins are: a) a VIP3 protein, and b) a Cry1F or Cry1A protein, preferably a VIP3 protein and a Cry1F protein. In one embodiment, such plants are used to delay or prevent insect resistance development to crop plants in insect populations of the fall armyworm (Spodoptera frugiperda).

Such transformed plants have advantages over plants transformed with a single insecticidal protein gene, or plants transformed with a Cry1F- and/or a Cry1A-encoding gene, especially with respect to the delay or prevention of resistance development in populations of the fall armyworm, against the insecticidal proteins expressed in such plants.

This invention also relates to a process for the production of transgenic plants, particularly corn, cotton, rice, soybean, and sugarcane, comprising two different insecticidal proteins that show no competition for binding to the binding sites in the midgut brush border of Spodoptera frugiperda larvae. Simultaneous expression in plants of chimeric genes encoding a VIP3 protein and a Cry1F or Cry1A protein, particularly a VIP3 and Cry1F protein, is particularly useful to prevent or delay resistance development of populations of fall armyworms against the insecticidal proteins expressed in such plants.

This invention further relates to a process for preventing or delaying the development of resistance in populations of Spodoptera frugiperda to transgenic plants expressing a Cry1A and/or a Cry1F protein, comprising providing such plants also with a gene expressing a VIP3 protein. Since such VIP3 protein and such Cry1A protein or such VIP3 protein and such Cry1F protein do not compete for binding sites in the midgut brush border of Spodoptera frugiperda larvae, these combinations are useful for securing long-lasting protection against said larvae.

This invention also relates to a method to control Spodoptera frugiperda insects in a region where populations of said insect species have become resistant to plants comprising a Cry1F and/or a Cry1A protein, comprising the step of sowing, planting or growing in said region, seeds or plants comprising a gene encoding a VIP3 protein. In one embodiment of the invention, said plants can also comprise (besides the gene encoding a VIP3 protein) a gene encoding another insecticidal protein which does not share binding sites with VIP3, Cry1F or Cry1A proteins in Spodoptera frugiperda.

### BACKGROUND OF THE INVENTION

Insect pests cause huge economic losses worldwide in crop production, and farmers face every year the threat of yield losses due to insect infestation. Genetic engineering of insect resistance in agricultural crops has been an attractive approach to reduce costs associated with crop-management and chemical control practices. The first generation of insect resistant crops have been introduced into the market since 1996, based on the expression in plants of insecticidal proteins isolated from the gram-positive soil bacterium Bacillus thuringiensis (abbreviated herein as “Bt”).

In contrast to the rapid development of insect resistance to some synthetic insecticides, so far development of insect resistance to plant-incorporated insecticidal proteins such as B. thuringiensis proteins has not evolved rapidly despite many years of use. This may be because of the insect resistance management programs which were used for such transgenic plants, such as the expression of a high dose level of protein for the main target insect(s), and the use of refuge areas (either naturally present or structured refuges) containing plants without such insecticidal proteins.

Procedures for expressing B. thuringiensis or other insecticidal protein genes in plants in order to render the plants insect-resistant are well known in the art and provide a new approach to insect control in agriculture which is at the same time safe, environmentally attractive and cost-effective. An important determinant for the continued success of this approach will be whether (or when) insects will be able to develop resistance to insecticidal proteins expressed in transgenic plants. In contrast to a foliar application, after which insecticidal proteins are typically rapidly degraded, the transgenic plants will exert a continuous selection pressure on the insects.

It is clear from laboratory selection experiments that a continuous selection pressure can lead to adaptation to insecticidal proteins, such as the B. thuringiensis Cry proteins, in insects.

While the insecticidal spectrum of different insecticidal proteins derived from Bt or other bacteria, such as the Cry or VIP3 proteins, can be different, the major pathway of their toxic action is common. All insecticidal proteins used in transgenic plants, for which the mechanism of action has been studied in at least one target insect, are proteolytically activated in the insect gut and interact with the midgut epithelium of sensitive species and cause lysis of the epithelial cells due to the fact that the permeability characteristics of the brush border membrane and the osmotic balance over this membrane are perturbed. In the pathway of toxic action of Cry proteins and VIP3 proteins, the binding of the toxin to receptor sites on the brush border membrane of these cells is an important feature (Hofmann et al., 1988; Lee et al., 2003). The binding sites are typically referred to as receptors, since the binding is saturable and with high affinity.

When two different insecticidal proteins share receptor binding sites in insects, they do not provide a good combination for insect resistance management purposes. Indeed, the most likely mechanism of resistance to insecticidal proteins such as Bt Cry proteins—and the only major mechanism found in field-developed insect resistance to Bt sprays so far—is receptor binding modification. Proteins that are highly similar in amino acid sequence often share receptor sites (e.g., the Cry1Ab and Cry1Ac proteins). But, even two different proteins having quite a different amino acid sequence may bind with high affinity to a common binding site in an insect species (such as the Cry1Ab and Cry1F proteins in this invention for S. frugiperda). Also, it has been found that two proteins that do not share binding sites in one insect species, may share a common binding site in another insect species (e.g., the Cry1Ac and Cry1Ba proteins were found to share a binding site in Chilo suppressalis in Fiuza et al. (1996) while they were found to bind to different binding sites in Plutella xylostella (Ballester et al. 1999)).

Based on data of a European corn borer population that was selected for resistance to Cry1F, it is said in published US patent application 20070006340 that a combination of Cry1F and Cry1Ab in corn is valuable in a insect resistance management strategy. No analysis was made on Spodoptera frugiperda insects in this publication.

### SUMMARY OF THE INVENTION

Provided herein is a method of controlling Spodoptera frugipera infestation in transgenic plants while securing a slower buildup of Spodoptera frugiperda insect resistance development to said plants, comprising expressing a combination of a) a VIP3 protein insecticidal to said insect species and b) a Cry1A or Cry1F protein insecticidal to said insect species, in said plants.

Also provided herein is a method for preventing or delaying insect resistance development in populations of the insect species Spodoptera frugiperda to transgenic plants expressing insecticidal proteins to control said insect pest, comprising expressing a VIP3 protein insecticidal to Spodoptera frugiperda in combination with a Cry1A or Cry1F protein insecticidal to Spodoptera frugiperda, particularly a Cry1F protein, in said plants.

In one embodiment of this invention, a method is provided to control Spodoptera frugiperda in a region where populations of said insect have become resistant to plants expressing a Cry1F or a Cry1A protein, comprising the step of sowing or planting in said region, plants expressing a VIP3 protein insecticidal to Spodoptera frugiperda.

Further provided herein is a method to control Spodoptera frugiperda in a region where populations of said insect have become resistant to plants expressing a VIP3 protein, comprising the step of sowing or planting in said region, plants expressing a Cry1F and/or Cry1A protein insecticidal to Spodoptera frugiperda.

Also provided in accordance with this invention is a method for obtaining plants expressing two different insecticidal proteins, wherein said proteins do not share binding sites in larvae of the species Spodoptera frugiperda as determined in competition binding experiments using brush border membrane vesicles of said insect larvae, comprising the step of obtaining plants comprising a plant-expressible chimeric gene encoding a VIP3 protein insecticidal to Spodoptera frugiperda and a plant-expressible chimeric gene encoding a Cry1A or Cry1F protein insecticidal to Spodoptera frugiperda, as well as such method wherein said plants are obtained by transformation of a plant with plant-expressible chimeric genes encoding said VIP3 and Cry1A of Cry1F proteins, and by obtaining progeny plants and seeds of said plant comprising said chimeric genes; or by the crossing of a parent plant comprising said VIP3-encoding chimeric gene with a parent plant comprising said Cry1A- or Cry1F-encoding chimeric gene, and obtaining progeny plants and seeds comprising said chimeric genes.

In another embodiment of this invention a method is provided for obtaining plants comprising chimeric genes expressing two different insecticidal proteins, wherein said proteins do not share midgut binding sites in larvae of the species Spodoptera frugiperda as can be determined in competition binding experiments using brush border membrane vesicles of said larvae, and wherein said proteins are: a) VIP3 protein insecticidal to Spodoptera frugiperda and b) a Cry1A or Cry1F protein insecticidal to Spodoptera frugiperda, particularly a Cry1F protein insecticidal to Spodoptera frugiperda; more particularly such method, wherein said plants are obtained by transformation of a plant with chimeric genes encoding said VIP3 and Cry1A of Cry1F proteins, and by obtaining progeny plants and seeds of said plant comprising said chimeric genes, or by crossing plants comprising a chimeric gene encoding said VIP3 protein with plants comprising a chimeric gene encoding said Cry1A or Cry1F protein, preferably said Cry1F protein, and obtaining progeny plants and seeds comprising said chimeric genes.

Also provided here is a method of sowing, planting, or growing plants protected against fall armyworms, comprising chimeric genes expressing two different insecticidal proteins, wherein said proteins do not share binding sites in larvae of the species Spodoptera frugiperda as determined in competition binding experiments using brush border membrane vesicles of said larvae, comprising the step of: sowing, planting, or growing plants comprising a chimeric gene encoding a VIP3 protein insecticidal to Spodoptera frugiperda and a chimeric gene encoding a Cry1A or Cry1F protein insecticidal to Spodoptera frugiperda, preferably a Cry1F protein insecticidal to Spodoptera frugiperda.

Also provided herein is the use of two different insecticidal proteins in transgenic plants to prevent or delay insect resistance development in populations of Spodoptera frugiperda, wherein said proteins do not share binding sites in the midgut of insects of said insect species, as can be determined by competition binding experiments, comprising expressing a VIP3 protein insecticidal to Spodoptera frugiperda and a Cry1F or Cry1A protein insecticidal to Spodoptera frugiperda in said transgenic plants, as well as the use of a chimeric gene encoding a VIP3 protein insecticidal to Spodoptera frugiperda and a chimeric gene encoding a Cry1F or Cry1A protein insecticidal to Spodoptera frugiperda, particularly a chimeric gene encoding a VIP3 protein insecticidal to Spodoptera frugiperda and a chimeric gene encoding a Cry1F protein insecticidal to Spodoptera frugiperda, for preventing or delaying insect resistance development in populations of the insect species Spodoptera frugiperda to transgenic plants expressing insecticidal proteins to control said insect pest.

In one embodiment herein is provided the use of a VIP3 protein insecticidal to Spodoptera frugiperda in combination with a Cry1A or Cry1F protein insecticidal to insects of said species, to prevent or delay resistance development of insects of said species to transgenic plants expressing heterologous insecticidal toxins, particularly when said use is by expression of said protein combination in plants.

Also provided herein is the use of plants comprising a VIP3 protein insecticidal to Spodoptera frugiperda in a region where populations of Spodoptera frugiperda have become resistant to plants comprising a Cry1F and/or Cry1A protein, wherein said use can comprise the sowing, planting or growing of plants comprising a VIP3 protein insecticidal to Spodoptera frugiperda in said region, as well as the use of plants comprising a Cry1F and/or Cry1A protein insecticidal to Spodoptera frugiperda in a region where S. frugiperda populations have become resistant to plants comprising a VIP3 protein, wherein said use can comprise the sowing, planting or growing of plants comprising a Cry1F and/or Cry1A protein insecticidal to Spodoptera frugiperda in said region.

Also provided herein is the use of a chimeric gene encoding a VIP3 protein insecticidal to Spodoptera frugiperda and a chimeric gene encoding a Cry1A or Cry1F protein insecticidal to Spodoptera frugiperda, particularly a chimeric gene encoding a VIP3 protein insecticidal to Spodoptera frugiperda and a chimeric gene encoding a Cry1F protein insecticidal to Spodoptera frugiperda, in a method to obtain plants capable of expressing two different insecticidal proteins, wherein said proteins do not share binding sites in larvae of the species Spodoptera frugiperda as can be determined in competition binding experiments, such as by using brush border membrane vesicles of said insect larvae.

In one embodiment of this invention, the use of a chimeric gene encoding a VIP3 protein insecticidal to Spodoptera frugiperda is provided to obtain plants comprising two different insecticidal proteins, wherein said proteins do not share binding sites in larvae of the species Spodoptera frugiperda, as can be determined in competition binding experiments, such as by using brush border membrane vesicles of said insect larvae, wherein said VIP3 chimeric gene is present in plants also comprising a chimeric gene encoding a Cry1A or Cry1F protein insecticidal to Spodoptera frugiperda.

In one embodiment, this use includes the obtaining of plants comprising such different insecticidal proteins by transformation of a plant with chimeric genes encoding said VIP3 and Cry1A of Cry1F proteins, and by obtaining progeny plants and seeds of said plant comprising said chimeric genes, and the obtaining of plants comprising such different insecticidal proteins by crossing plants comprising a chimeric gene encoding said VIP3 protein with plants comprising a chimeric gene encoding said Cry1A or Cry1F protein.

In accordance with the invention, the VIP3 chimeric gene used in the above processes and uses encodes a VIP3A protein such as VIP3Aa1, VIP3Af1, VIP3Aa19 or VIP3Aa20 protein, or is a chimeric gene comprising a VIP3 coding region selected from the group consisting of: the VIP3 coding region contained in corn event MIR162 of USDA APHIS petition 07-253-01p (WO 2007/142840), the VIP3 coding region contained in cotton event COT102 of USDA APHIS petition 03-155-01p (WO 2004/039986), the VIP3 coding region contained in cotton event COT202 described in WO 2005/054479, and the VIP3 coding region contained in cotton event COT203 described in WO 2005/054480.

In accordance with the invention, the Cry1F chimeric gene used in the above uses or processes encodes a Cry1Fa protein, and particularly is a chimeric gene comprising a Cry1F coding region selected from the group consisting of: the Cry1F coding region contained in corn event TC1507 of USDA APHIS petition 00-136-01p (WO 2004/099447), the Cry1F coding region contained in corn event TC-2675 of USDA APHIS petition 03-181-01p or corn event TC-2675 of USDA APHIS petition 03-181-01p, and the Cry1F coding region contained in cotton event 281-24-236 event of USDA APHIS petition 03-036-01p (the Cry1F gene-containing event of WO 2005/103266).

In accordance with the invention, the Cry1A chimeric gene as used in the above processes or uses encodes a Cry1Ab, Cry1A.105 or Cry1Ac protein, and particularly is a chimeric gene comprising a coding region selected from the group consisting of: the Cry1Ab coding region contained in corn event MON810 of USDA APHIS petition 96-017-01p (U.S. Pat. No. 6,713,259), the Cry1Ab coding region contained in corn event Bt11 of USDA APHIS petition 95-195-01p (U.S. Pat. No. 6,114,608), the Cry1Ab coding region contained in cotton event COT67B of USDA APHIS petition 07-108-01p, the Cry1Ac coding region contained in cotton event 3006-210-23 of USDA APHIS petition 03-036-02p (WO 2005/103266), the Cry1Ac coding region contained in cotton event 531 of USDA APHIS petition 94-308-01p (or the Cry1A gene event of WO 2002/100163), and the Cry1A.105 coding region contained in corn event MON89034 of USDA APHIS petition 06-298-01p (the Cry1A.105 coding region described in WO 2007/140256, encoding the protein of SEQ ID No.7).

In accordance with this invention, in the above uses or methods the VIP3, Cry1F or Cry1A chimeric genes are the chimeric genes contained in any one of the above corn or cotton events.

In one embodiment in the invention, the VIP3 protein used is a VIP3A protein insecticidal to Spodoptera frugiperda, such as the VIP3Aa1, VIP3Af1, VIP3Aa19 or VIP3Aa20 proteins described herein, but also any protein comprising an insecticidal fragment or functional domain thereof, as well as any protein insecticidal to Spodoptera frugiperda with a sequence identity of at least 70% with the VIP3Aa1 protein of NCBI accession AAC37036, particularly with its smallest toxic fragment, or with the VIP3Af 1 protein of NCBI accession CAI43275, particularly with its smallest toxic fragment, as determined using pairwise alignments using the GAP program of the Wisconsin package of GCG.

In the uses or methods of the current invention, preferred plants, such as for stacking different chimeric genes in the same plants by crossing, are plants comprising any one of the above corn or cotton events, as well as their progeny or descendants comprising said VIP3 and Cry1 protein-encoding chimeric genes.

Plants used in the above embodiments include plants of any plant species significantly damaged by fall armyworms, but particularly include corn, cotton, rice, soybean and sugarcane.

The invention also provides for the use, the sowing, planting or growing of a refuge area with plants not comprising a Cry1 or VIP protein insecticidal to Spodoptera frugiperda, such as by sowing, planting or growing such plants in the same field or in the vicinity of the plants comprising the VIP3 and Cry1 protein described herein.

Also provided herein are the above uses or processes wherein the plants express the VIP3 and/or Cry1F or Cry1A proteins at a high dose for S. frugiperda.

Further provided herein are plants or seeds comprising at least a VIP3A and a Cry1A or Cry1F transgene each encoding a different protein insecticidal to S. frugiperda which proteins bind specifically to binding sites in the midgut of such insects, wherein said proteins do not compete for the same binding sites in such insects, and wherein said VIP3A protein is a protein comprising the smallest toxic fragment of a VIP3Aa or VIP3Af protein, and said Cry1A or Cry1F protein is a protein comprising the smallest toxic fragment of a Cry1Ab, Cry1A.105, or Cry1Ac, or Cry1Fa protein, particularly such plants or seeds, which are corn or cotton plants or seeds containing a combination of at least 2 or at least 3 different transformation events selected from the group consisting of: for corn: corn event MON89034, corn event MIR162, corn event TC1507, corn event TC-2675, corn event Bt11, or corn event MON810; for cotton: cotton event COT102, cotton event COT202, cotton event COT203, cotton event T342-142, cotton event 1143-14A, cotton event 1143-51 B, cotton event CE44-69D, cotton event CE46-02A, cotton event COT67B, cotton event 15985, cotton event 3006-210-23, cotton event 531, cotton event EE-GH5, and cotton Event 281-24-236.

Also provided herein is a method for obtaining regulatory approval for planting or commercialization of plants expressing proteins insecticidal to S. frugiperda, comprising the step of referring to, submitting or relying on insect assay binding data showing that VIP3A proteins do not compete with binding sites for Cry1A or Cry1F proteins in such insect species, as well as a method for obtaining a reduction in structured refuge area containing plants not producing any Bt protein insecticidal to S. frugiperda in a field, such method comprising the step of referring to, submitting or relying on insect assay binding data showing that VIP3A proteins do not compete with binding sites for Cry1A or Cry1F proteins in such insect species, particularly such methods, wherein said VIP3A protein is a protein comprising the smallest toxic fragment of a VIP3Aa or VIP3Af protein and wherein said Cry1A or Cry1F protein is a protein comprising the smallest toxic fragment of a Cry1Ac, Cry1Ab, Cry1A.105, or Cry1F protein, such as any one of the proteins encoded by the transgenic events identified in the description.

Also included herein is a field of insect-resistant transgenic plants controlling S. frugiperda insects, wherein said field has a structured refuge area of less than 20%, of less than 15%, of less than 10%, or of less than 5%, or has no structured refuge area, wherein said plants express a combination of a VIP3Aa or VIP3Af protein insecticidal to S. frugiperda insects, and a Cry1A or Cry1F protein insecticidal to S. frugiperda insects, particularly a VIP3Aa1, VIP3Af1, VIP3Aa19 or VIP3Aa20 protein and a Cry1Ab, Cry1A.105, Cry1Ac or Cry1Fa protein insecticidal to S. frugiperda insects, preferably a VIP3Aa, a Cry1Ab or Cry1A.105 and a Cry1F protein, insecticidal to S. frugiperda insects.

Further provided herein is a method of controlling Spodoptera frugipera infestation in transgenic plants while securing a slower buildup of Spodoptera frugiperda insect resistance development to said plants, comprising expressing in said plants a Cry1A protein insecticidal to said insect species with another protein which is insecticidal to Spodoptera frugiperda, which does not share receptor binding sites in the midgut of such insect species with said Cry1A protein, and which is not a Cry1F protein. Also provided herein is a method of controlling Spodoptera frugipera infestation in transgenic plants while securing a slower buildup of Spodoptera frugiperda insect resistance development to said plants, comprising expressing in said plants a Cry1F protein insecticidal to said insect species with another protein which is insecticidal to Spodoptera frugiperda, which does not share receptor binding sites in the midgut of such insect species with said Cry1F protein, and which is not a Cry1A protein. In one embodiment, two different insecticidal proteins do not share receptor binding sites in the midgut of such insect species if there is no biological significant competition for the different binding sites between the two different proteins in standard binding assays using midgut brush border membrane vesicles of an insect.

Also provided herein is a method for preventing or delaying insect resistance development in populations of the insect species Spodoptera frugiperda to transgenic plants expressing insecticidal proteins to control said insect pest, comprising expressing in said plants a Cry1A protein insecticidal to Spodoptera frugiperda in combination with another protein which is insecticidal to Spodoptera frugiperda and which does not share receptor binding sites in the midgut of such insect species, and which is not a Cry1F protein.

### DETAILED DESCRIPTION OF THE INVENTION

Because of the success and the increasing number of plants comprising introduced insecticidal proteins such as Bt Cry or VIP3 proteins, resistance management is even more important now than in the past.

Spodoptera frugiperda (or S. frugiperda) or the fall armyworm is considered a significant pest in the USA and a main pest in South and Central America, and it can cause major damage to crop plantings, with production losses of up to 38%. It attacks a variety of plants, but important crop plants attacked are corn, cotton, rice, soybean, and sugarcane.

In the current invention it has been found that a Cry1F protein competes for the same midgut binding site as Cry1Ab in Spodoptera frugiperda, and hence a combination of these two proteins in the same plant is not a good approach for resistance management of Spodoptera frugiperda insects.

In the current invention, receptor binding analysis showed that in this insect species, VIP3 proteins do not show competition for the Cry1F or Cry1A receptor, making it most interesting to combine in the same plant a VIP3 protein with a Cry1F or Cry1A protein, preferably a VIP3 protein and a Cry1F protein, to prevent or delay the development of insect resistance to Spodoptera frugiperda. In one embodiment the VIP3 protein is a VIP3Aa (e.g., VIP3Aa19 or VIP3Aa20) or a VIP3Af protein. This approach should ideally be part of a general approach for insect resistance management including, where necessary, refuge areas and the expression of the proteins at a high dose for the target insect.

The binding sites which are referred to herein only refer to the specific binding sites for insecticidal proteins toxic to S. frugiperda, such as the VIP3Aa or Cry1Fa proteins. These are the binding sites to which a protein binds specifically, i.e., for which the binding of a labeled ligand (such as a VIP3 of Cry1Fa protein), to its binding site, can be displaced (or competed for) by an excess of non-labeled homologous ligand (a VIP3 or Cry1Fa protein, respectively). The terms binding site or receptor are used interchangeably herein and are equivalent.

It is important when combining different insecticidal proteins in plants with the aim to delay or decrease insect resistance development of a target insect species, to check experimentally (i.e., by performing binding assays) in the target insect species if a proposed combination of different insecticidal proteins shares binding sites in the midgut of the target insect. Only when there is no (biologically significant) competition for the different binding sites between two different insecticidal proteins, is it useful to combine such proteins from an insect resistance management perspective. As used herein, competition is not considered biologically significant if the competition takes place only at very high concentrations of the heterologous competitor (e.g., if 100 nM of the unlabeled heterologous competitor displaces only a minimal amount of bound labeled ligand (e.g., about 25% or less of the specific binding of the labeled ligand)).

The methods and techniques for testing sharing of binding sites to insect larvae for two different insecticidal proteins are well known in the art (see, e.g., Van Rie et al., 1989, Ferré et al., 1991). At first, one determines a pair of insecticidal proteins which are both insecticidal to the target insect, here S. frugiperda. Brush border membrane vesicles (BBMV) are prepared from the midguts of Spodoptera frugiperda using known procedures (see, e.g., Wolfersberger et al. 1987), and the specific binding of purified labeled protein (such as a VIP3 or Cry1 protein) to such BBMV is analyzed. Homologous competition assays are done to determine if the binding is specific (herein an excess of the same unlabeled protein is used as competitor for the labeled ligand), and heterologous competition assays are done to determine if another protein competes for the same binding site in these BBMV (herein an excess of a different, unlabeled protein is used as competitor for the labeled ligand). In homologous competition assays, the binding is specific if the binding of labeled protein is competed for (or displaced by) the unlabeled protein (i.e., the homologous competitor)—the binding which is not displaced or competed for by homologous ligand is considered non-specific binding. Labeling of the proteins, such as the VIP3 or Cry1 proteins used in this invention, can be done by the well known techniques of biotin-labeling, fluorescent labeling, or by radioactive labeling, such as by using Na¹²⁵Iodine (using known methods, e.g., Chloramine-T method).

In accordance with this invention, a “nucleic acid sequence” refers to a DNA or RNA molecule in single or double stranded form, preferably a DNA or RNA, particularly a DNA, encoding any of the proteins used in this invention. An “isolated nucleic acid sequence”, as used herein, refers to a nucleic acid sequence which is no longer in the natural environment where it was isolated from, e.g., the nucleic acid sequence in another bacterial host or in a plant nuclear genome.

As used herein “heterologous” proteins, such as when referring to the use of heterologous insecticidal proteins in plants, refers to proteins not present in such organism in nature, particularly to proteins encoded by transgenes introduced into the genome of plants, wherein such proteins are derived from bacterial proteins.

In accordance with this invention, the terms “protein” or “polypeptide” are used interchangeably to refer to a molecule consisting of a chain of amino acids, without reference to any specific mode of action, size, three-dimensional structures or origin. Hence, a fragment or portion of a protein used in the invention is still referred to herein as a “protein”. An “isolated protein”, as used herein, refers to a protein which is no longer in its natural environment. The natural environment of the protein refers to the environment in which the protein could be found when the nucleotide sequence encoding it was expressed and translated in its natural environment, i.e., in the environment from which the nucleotide sequence was isolated. For example, an isolated protein can be present in vitro, or in another bacterial host or in a plant cell or it can be secreted from another bacterial host or from a plant cell.

As used herein, “insecticidal protein” should be understood as an intact protein or a part thereof which has insecticidal activity, particularly insecticidal to Spodoptera frugiperda larvae. This can be a naturally-occurring protein or a chimeric protein comprising parts of different insecticidal proteins, or can be a variant having substantially the amino acid sequence of a bacterial protein but modified in some (e.g., 1, 2, 3, 4, 5, 6, 7, 8, 9 or 10) amino acids. In this regard, such an insecticidal protein can be a VIP or a Cry protein derived from Bt or other bacterial strains.

As used herein, “protoxin” should be understood as the primary translation product of a full-length gene encoding an insecticidal protein, before any cleavage has occurred in the midgut. Typically, a VIP3 protoxin has a molecular weight of about 88 kD, a Cry1F or Cry1A protoxin has a molecular weight of about 130-140 kD.

As used herein, “toxin” or “smallest toxic fragment” should be understood as that part of an insecticidal protein, such as a VIP3 or Cry1F or Cry1A protein, which can be obtained by trypsin digestion or by proteolysis in (target insect, e.g., Spodoptera frugiperda) midgut juice, and which has insecticidal activity. Typically, a VIP3 or Cry toxin or smallest toxic fragment has a molecular weight of about 60-65 kD. In one embodiment, the smallest toxic fragment of a Cry1F protein as used herein is a protein from amino acid position 29 to amino acid position 604 of any one of SEQ ID No. 1, 9 or 10, and the smallest toxic fragment of a Cry1Ac protein as used herein is a protein from amino acid position 29 to amino acid position 607 in any one of SEQ ID No. 6 or 11, and the smallest toxic fragment of a Cry1Ab protein is a protein from amino acid position 29 to amino acid position 607 in SEQ ID No. 8.

As used herein, a “VIP3 protein” or “VIP3”, refers to a protein insecticidal to Spodoptera frugiperda larvae, and which is any one of the VIP3 proteins listed in Table 2 or in Crickmore et al. (2008) on the VIP nomenclature website at: www.lifesci.susx.ac.uk/home/Neil_Crickmore/Bt/VIP.html, or any protein comprising the smallest toxic fragment of any one of these proteins, particularly any protein comprising an amino acid sequence differing in less than 10, 9, 8, 7, 6, 5, 4, or less than 3 amino acids from the smallest toxic fragment of any VIP3 protein, such as any of the above proteins in the Crickmore list or any protein in a publication with at least 70% sequence identity to a known VIP3 protein. In one embodiment, this is a VIP3A protein insecticidal to Spodoptera frugiperda, such as a VIP3Aa1 protein of SEQ ID No. 2, a VIP3Af1 protein of SEQ ID No. 3, a VIP3Aa19 protein of SEQ ID No. 4 or a VIP3Aa20 protein of SEQ ID No. 5 (described in said nomenclature website and below), but also any insecticidal fragments thereof, or proteins with a sequence identity of at least 70%, particularly at least 75%, 80%, 85%, 90%, 95%, 96%, 97%, 98% or 99% at the amino acid sequence level with the VIP3Aa1 protein of NCBI accession AAC37036 or SEQ ID No. 2, the VIP3Af1 protein of NCBI accession CAI43275 or SEQ ID No. 3, the VIP3Aa19 protein of SEQ ID No. 4, or the VIP3Aa20 protein of SEQ ID No. 5, particularly with their smallest toxic fragment, as determined using pairwise alignments using the GAP program of the Wisconsin package of GCG (Madison, Wis., USA, version 10.2). The GAP program is used with the following parameters for the amino acid sequence comparisons: the ‘blosum62’ scoring matrix, a ‘gap creation penalty (or ‘gap weight’) of 8 and a ‘gap extension penalty’ (or ‘length weight’) of 2. In one embodiment, a VIP3 protein as used herein, is a VIP3A protein such as the VIP3Aa1 protein described in Estruch et al. (1996, NCBI accession AAC37036, SEQ ID No. 2), or any VIP3A protein, insecticidal to S. frugiperda, described in the above VIP nomenclature website or in the NCBI database, as well as a VIP3A protein insecticidal to Spodoptera frugiperda selected from the group of: VIP3Ab, VIP3Ac, VIP3Ad, VIP3Ae, VIP3Af, VIP3Ag, or VIP3Ah, particularly the VIP3Af1, VIP3Ad1 or VIP3Ae1 proteins (NCBI accessions CAI43275, CAI43276, and CAI43277, respectively) and insecticidal fragments, hybrids or variants thereof. Of course, besides the naturally-occurring protein, and proteins comprising an insecticidal fragment thereof also hybrid or chimeric proteins made from VIP3 proteins retaining insecticidal activity to S. frugiperda are included herein, such as the chimeric VIP3AcAa protein described in Fang et al. (2007), as well as protein mutants or equivalents differing in some amino acids but retaining most or all of the S. frugiperda toxicity of the parent molecule; such as VIP3 protein variants having some, preferably 5-10, particularly less than 5, amino acids added, replaced or deleted, preferably in the part corresponding to the smallest toxic fragment, without significantly changing the Spodoptera frugiperda insecticidal activity of the protein, e.g., such as the VIP3Aa19 protein (NCBI accession ABG20428) introduced in cotton plants (e.g., in plants containing event COT102 described in WO 2004/039986, or in USDA APHIS petition for non-regulated status 03-155-01p) or the VIP3Aa20 protein (NCBI accession ABG20429, SEQ ID NO: 2 in WO 2007/142840) introduced in corn plants (e.g., event MIR162, USDA APHIS petition for non-regulated status 07-253-01p), or the VIP3A proteins produced in the COT202 or COT203 cotton events (WO 2005/054479 and WO 2005/054480, respectively).

Also, in a VIP3 protein of the current invention any putative native (bacterial) secretion signal peptide can be deleted or can be replaced by a Met amino acid or Met-Ala dipeptide, or by an appropriate signal peptide, such as a chloroplast transit peptide. Putative signal peptides can be detected using computer based analysis, using programs such as the program Signal Peptide search (SignalP V1.1 or 2.0), using a matrix for prokaryotic gram-positive bacteria and a threshold score of less than 0.5, especially a threshold score of 0.25 or less (Von Heijne, Gunnar, 1986 and Nielsen et al., 1996).

A “Cry1F protein” or “Cry1F”, as used herein, includes any protein comprising the smallest toxic fragment of the amino acid sequence of a Cry1F protein retaining toxicity to Spodoptera frugiperda, such as the protein in NCBI accession AAA22347 or SEQ ID No. 1, 9 or 10. This includes hybrid or chimeric proteins comprising this smallest toxic fragment, or at least one of the structural domains, preferably at least 2 of the 3 structural domains, of a Cry1F protein, such as the proteins in SEQ ID No. 9 or 10 which are produced in corn and cotton plants, respectively, containing a cry1F transgene. Also included in this definition are variants of the amino acid sequence in NCBI accession AAA22347 or SEQ ID No. 1, 9 or 10, such as amino acid sequences having a sequence identity of at least 90%, 95%, 96%, 97%, 98% or 99% to the Cry1F protein of NCBI accession AAA22347 or SEQ ID No. 1, 9 or 10 at the amino acid sequence level, as determined using pairwise alignments using the GAP program of the Wisconsin package of GCG (Madison, Wis., USA, version 10.2), particularly such identity is with the part corresponding to the smallest toxic fragment. The GAP program is used with the following parameters for the amino acid sequence comparisons: the ‘blosum62’ scoring matrix, a ‘gap creation penalty’ (or ‘gap weight’) of 8 and a ‘gap extension penalty’ (or ‘length weight’) of 2. Preferably proteins having some, preferably 5-10, particularly less than 5, amino acids added, replaced or deleted without significantly changing the Spodoptera frugiperda insecticidal activity of the protein, such as a Cry1F protein with one or more conservative amino acid substitutions for cloning purposes, are included in this definition. A Cry1F protein, as used herein, includes the protein encoded by the Cry1F genes in Cry1F Cotton Event 281-24-236 (WO 2005/103266, see USDA APHIS petition for non-regulated status 03-036-01p, see the Cry1F.281-24-236 protein in SEQ ID No. 10), or in corn events TC1507 or TC-2675 (U.S. Pat. No. 7,288,643, WO 2004/099447, USDA APHIS petitions for non-regulated status 00-136-01p and 03-181-01p, see the Cry1F.6275 protein in SEQ ID No. 9), particularly any protein comprising the smallest toxic fragment of any one of such Cry1F proteins as defined above.

In the current invention, it has been found that a Cry1F protein competes for the same binding sites as the Cry1Ab protein in S. frugiperda, and that these binding sites are different (not shared) from the binding sites of VIP3A proteins in Spodoptera frugiperda. Since it has already been reported that Cry1Ab and Cry1Ac share the same binding sites in Spodoptera frugiperda (e.g., Rang et al. 2004), it is clear that both Cry1Ab and Cry1Ac bind to a binding site that is different from the binding site of VIP3 in S. frugiperda. Although Cry1A proteins generally have a lower activity to fall armyworms compared to the Cry1F or VIP3 proteins tested, they are the first and amongst the most widely used Cry proteins in plants, and since they do not share binding sites with VIP3 proteins, they can also be useful for insect resistance management, certainly if the plants can provide for high levels of expression of the Cry1A protein. Some Cry1A proteins have a higher intrinsic activity to S. frugiperda, and these are a more preferred Cry1A proteins in this invention, e.g., the Cry1A.105 protein as described below or in SEQ ID No. 7 herein, or similar chimeric or hybrid Cry1A proteins with increased fall armyworm activity, as described in U.S. Pat. No. 6,962,705 or U.S. Pat. No. 7,070,982. When there is a choice between a Cry1F and a Cry1Ab, Cry1A.105, or Cry1Ac protein to combine (by crossing plants expressing a single insecticidal protein or by transformation) with a VIP3 protein in a given plant species, a Cry1F or Cry1A.105 protein will be the better choice to delay or prevent resistance development to Spodoptera frugiperda, given their higher toxicity to this insect species.

A “Cry1A” protein, as used herein, refers to a Cry1Ac, Cry1A.105 or Cry1Ab protein, and includes any protein comprising the smallest toxic fragment of the amino acid sequence of a Cry1Ac, Cry1A.105 or Cry1Ab protein retaining toxicity to Spodoptera frugiperda, such as the smallest toxic fragment of the protein in NCBI accession AAA22331 (Cry1Ac) or SEQ ID No. 6 or 11, the smallest toxic fragment of the protein of SEQ ID No. 7 (Cry1A.105), or the smallest toxic fragment of the protein of NCBI accession CAA28405 (Cry1Ab) or of SEQ ID No. 8. This includes hybrid or chimeric proteins comprising this smallest toxic fragment or at least one of the structural domains, preferably at least 2 of the 3 structural domains, of a Cry1A protein such as Cry1Ab or Cry1Ac, e.g., the chimeric or hybrid Cry1A proteins with increased fall armyworm activity, as described in U.S. Pat. No. 6,962,705 or U.S. Pat. No. 7,070,982. Also included in this definition are variants of the amino acid sequence in NCBI accession AAA22331 (Cry1Ac1) or in SEQ ID No. 6 or 11, or in NCBI accession CAA28405 (Cry1Ab) or SEQ ID No. 8 or variants of the Cry1A.105 protein of SEQ ID No. 7, such as amino acid sequences having a sequence identity of at least 90%, 95%, 96%, 97%, 98% or 99% at the amino acid sequence level with such a Cry1Ac, Cry1A.105 or Cry1Ab protein, particularly in the part corresponding to the smallest toxic fragment, as determined using pairwise alignments using the GAP program of the Wisconsin package of GCG (Madison, Wis., USA, version 10.2), with the smallest toxic fragment of a Cry1A protein. The GAP program is used with the following parameters for the amino acid sequence comparisons: the ‘blosum62’ scoring matrix, a ‘gap creation penalty’ (or ‘gap weight’) of 8 and a ‘gap extension penalty’ (or ‘length weight’) of 2. Preferably proteins having some, preferably 5-10, particularly less than 5, amino acids added, replaced or deleted without significantly changing the Spodoptera frugiperda insecticidal activity of the protein, such as a Cry1A protein with one or more conservative amino acid substitutions (e.g., for gene cloning purposes), are included in this definition.

Examples of Cry1A proteins for use in this invention include the Cry1Ab protein encoded by SEQ ID NO:3 of U.S. Pat. No. 6,114,608, particularly the Cry1Ab protein encoded by the cry1Ab coding region in corn event MON810 (U.S. Pat. No. 6,713,259), USDA APHIS petition for non-deregulated status 96-017-01p and extensions thereof), the Cry1Ab protein encoded by the cry1Ab coding region in corn event Bt11 (USDA APHIS petition for non-deregulated status 95-195-01p, U.S. Pat. No. 6,114,608), the Cry1Ac protein encoded by the transgene in cotton event 3006-210-23 (U.S. Pat. No. 7,179,965, WO 2005/103266, USDA APHIS petition for non-deregulated status 03-036-02p, see SEQ ID No. 11), the Cry1Ab protein encoded by the cry1Ab coding region in cotton event COT67B (USDA APHIS petition for non-deregulated status 07-108-01p), the Cry1A.105 protein encoded by the Cry1A transgene in corn event MON89034 (USDA APHIS petition for non-regulated status 06-298-01p, WO 2007/140256, SEQ ID NO: 2 or 4 in WO 2007/027777, or SEQ ID No. 7 herein), the Cry1Ac-like protein encoded by the hybrid cry1Ac coding region in cotton event 15985 or cotton event 531, 757, or 1076 (USDA APHIS petition for non-regulated status 94-308-01p, the chimeric Cry1Ac protein encoded by the cryIA cotton event of WO 2002/100163), or a protein differing from any of these proteins in 1, 2, 3, 4, or 5 amino acids. In one embodiment of this invention, a Cry1Ab or a Cry1A.105 protein from this above list is used, such as the protein of SEQ ID No. 8 or any protein comprising the toxic fragment thereof, or the protein of SEQ ID No. 7 or any protein comprising the toxic fragment thereof.

It is well known that Bt Cry proteins such as Cry1F and Cry1A proteins are expressed as protoxins in their native host cells (Bacillus thuringiensis), which are converted into the toxin form by proteolysis in the insect gut. A Cry1F or Cry1A protein, as used herein, refers to either the full protoxin or the toxin, or any intermediate form with insecticidal activity. In one embodiment, a Cry1F protein includes a protein comprising the amino acid sequence of NCBI accession AAA22347 or any one of SEQ ID No. 1, 9 or 10, or any protein comprising the amino acid sequence from amino acid position 29 to amino acid position 604 in any one of SEQ ID No 1, 9 or 10, and a Cry1A protein includes a protein comprising the amino acid sequence of NCBI accession AAA22331 (Cry1Ac1) or of SEQ ID No. 6 or 11 from amino acid position 29 to 607, or comprising the amino acid sequence of NCBI accession CAA28405 (Cry1Ab) or SEQ ID No. 8 from amino acid position 29 to 607, or comprising the amino acid sequence of SEQ ID No. 7 (Cry1A.105) from amino acid position 29 to 612.

A “Cry1” protein, as used herein, refers to a Cry1F or Cry1A protein as defined above. A VIP3 or cry1 “gene” or “DNA”, as used herein, refers to a DNA encoding a VIP3 or Cry1 protein in accordance with this invention. A gene can be naturally occurring, artificial (modified) or synthetic in whole or in part.

The term “event”, as used herein, refers to a specific integration of one or more transgenes at a specific location in the plant genome, which can be considered as a part of DNA containing the inserted sequences and the flanking plant sequences. Such an event can be crossed into many other plants of the same species by normal breeding.

As used herein “comprising” is to be interpreted as specifying the presence of the stated features, integers, steps or components as referred to, but does not preclude the presence or addition of one or more features, integers, steps or components, or groups thereof. Thus, the term “DNA/protein comprising the sequence or region X”, as used herein, refers to a DNA or protein including or containing at least the sequence or region X, so that other nucleotide or amino acid sequences can be included at the 5′ (or N-terminal) and/or 3′ (or C-terminal) end, e.g. (the nucleotide sequence of) a transit peptide, and/or a 5′ or 3′ leader sequence.

A VIP3 or Cry1 protein-encoding “chimeric gene”, as used herein, refers to a VIP3 or Cry1-encoding DNA (or coding region) having 5′ and/or 3′ regulatory sequences, at least a 5′ regulatory sequence or promoter, different from the naturally-occurring bacterial 5′ and/or 3′regulatory sequences which drive the expression of the VIP3 or Cry1 protein in its native host cell, e.g., a VIP3 or cry1 DNA operably-linked to a plant-expressible promoter (including a promoter active in chloroplasts, other plastids or mitochondria) such that said chimeric gene can be expressed in the plants containing it. The chimeric gene need not be expressed the entire time or in every cell of the plant, e.g., expression can be induced by insect feeding or wounding using a wound-induced promoter, or expression can be localized in those plant parts mostly attacked by insects such as Spodoptera frugiperda insects or most valuable for the grower or farmer, e.g., the leaves and ears of a corn plant, or the leaves and bolls of cotton plants, or the leaves and pods of soybean plants. Hence, a plant expressing a VIP3, Cry1F or Cry1A protein as used herein refers to a plant containing the necessary plant-expressible chimeric gene encoding such a protein, so that the protein is expressed in the relevant tissues or at the relevant time periods, which need not be in all plant tissues or need not be at all time periods.

For the purpose of this invention the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other is regarded as a position with non-identical residues. To calculate sequence identity between two sequences for the purpose of this invention, the GAP program, which uses the Needleman and Wunsch algorithm (1970) and which is provided by the Wisconsin Package, Version 10.2, Genetics Computer Group (GCG), 575 Science Drive, Madison, Wis. 53711, USA, is used. The GAP parameters used are a gap creation penalty=50 (nucleotides)/8 (amino acids), a gap extension penalty=3 (nucleotides)/2 (amino acids), and a scoring matrix “nwsgapdna” (nucleotides) or “blosum62” (amino acids).

GAP uses the Needleman and Wunsch global alignment algorithm to align two sequences over their entire length, maximizing the number of matches and minimizes the number of gaps. The default parameters are a gap creation penalty=50 (nucleotides)/8 (proteins) and gap extension penalty=3 (nucleotides)/2 (proteins). For nucleotides the default scoring matrix used is “nwsgapdna” and for proteins the default scoring matrix is “blosum62” (Henikoff & Henikoff, 1992).

DNAs included herein as a VIP3 or Cry1 DNA are those DNAs that encode a VIP3 or Cry1 protein, or a variant or hybrid thereof, insecticidal to S. frugiperda, and that hybridizes under stringent hybridization conditions to a DNA that can encode a VIP3 or Cry1 protein. “Stringent hybridization conditions”, as used herein, refers particularly to the following conditions: immobilizing the relevant DNA on a filter, and prehybridizing the filters for either 1 to 2 hours in 50% formamide, 5% SSPE, 2×Denhardt's reagent and 0.1% SDS at 42° C. or 1 to 2 hours in 6×SSC, 2×Denhardt' s reagent and 0.1% SDS at 68° C. The denatured (Digoxigenin- or radio-) labeled probe is then added directly to the prehybridization fluid and incubation is carried out for 16 to 24 hours at the appropriate temperature mentioned above. After incubation, the filters are then washed for 30 minutes at room temperature in 2×SSC, 0.1% SDS, followed by 2 washes of 30 minutes each at 68° C. in 0.5×SSC and 0.1% SDS. An autoradiograph is established by exposing the filters for 24 to 48 hours to X-ray film (Kodak XAR-2 or equivalent) at −70° C. with an intensifying screen. [20×SSC=3M NaCl and 0.3M sodiumcitrate; 100×Denhart's reagent=2% (w/v) bovine serum albumin, 2% (w/v) Ficoll™ and 2% (w/v) polyvinylpyrrolidone; SDS=sodium dodecyl sulfate; 20×SSPE=3.6M NaCl, 02M Sodium phosphate and 0.02M EDTA pH7.7]. Of course, equivalent conditions and parameters can be used in this process while still retaining the desired stringent hybridization conditions.

“Insecticidal activity” of a protein, as used herein, means the capacity of a protein to kill insects when such protein is fed to insects, preferably by expression in a recombinant host such as a plant. It is understood that a protein has insecticidal activity if it has the capacity to kill the insect during at least one of its developmental stages, preferably the larval stage.

A population of insect species that “has developed resistance” or “has become resistant” to plants expressing an insecticidal protein (which plants formerly controlled or killed populations of said insect), as used herein, refers to the detection of repeated, significant unacceptable yield damage in such plants, caused by such insect population as compared to the level of yield damage of such plants by the same insect species when such plants were first introduced. This has to be confirmed to check that the plants are indeed producing the insecticidal protein (i.e., they are not non-transgenic plants), and that members of this insect population indeed need a higher amount of insecticidal protein to be controlled or killed. In other words, such plants to which an insect population has become resistant no longer produce an insect-controlling amount (as defined herein) or are no longer insecticidal for such insect species population. As such, “insect resistance development” as used herein, refers to the increased plant damage that is detected. In one embodiment, insect resistance of an insect species population is readily observed if insects from such population can complete their life cycle on such plants, and continue to damage the plants instead of being arrested in their growth and feeding habits because of the insecticidal proteins produced in such plants—in an extreme form of insect resistance such plant can be as damaged as conventional untransgenic plants with the same genetic background by an insect attack. In one embodiment, the binding to Cry1 or VIP3 proteins to such resistant insects can be analyzed in (standard) competition binding assays using BBMV of S. frugiperda, to confirm that resistance is due to binding site modification. “Fall armyworm”, or “S. frugiperda”, as used herein, refers to Spodoptera frugiperda (JE Smith), an important Lepidopteran pest insect.

“Insect-controlling amounts” of a protein, as used herein, refers to an amount of protein which is sufficient to limit damage on a plant, caused by insects (e.g. insect larvae) feeding on such plant, to commercially acceptable levels, e.g. by killing the insects or by inhibiting the insect development, fertility or growth in such a manner that they provide less damage to a plant and plant yield is not significantly adversely affected.

A “structured refuge” as used herein, refers to an area of non-Bt fields or non-Bt parts of fields in or adjacent to a Bt-crop that is planted to the same crop, particularly a part of the field or land of a grower or farmer that is otherwise planted with Bt-plants, but which is planted with plants not containing a Bt transgene (as compared to using weeds or other non-Bt plants around a farmer's fields, which is known as an unstructured or a natural refuge). Also included herein as structured refuge is a non-Bt portion of a grower's field or set of fields (planted with an insecticidal Bt-protein producing crop) that provides for the production of susceptible (SS) insects that may randomly mate with rare resistant (RR) insects surviving the Bt-protein producing crop to produce susceptible heterozygotes (RS). A structured refuge can be planted in the same field as a Bt-crop, or adjacent to it, but is usually planted within 0.25, within 0.5 or within 0.75 or 1 mile from the Bt-crop field, but can be of the size and distance from a Bt-field as is required or desired by national regulatory authorities. A structured refuge may, e.g., be required on 20% or 50% of the field, depending, e.g., on what crop you plant, how effective that crop kills the target insects, and which and how much other Bt-crops are grown in the same area. Seed mixes of Bt- and non-Bt-producing plants of the same crop or plant species are not yet allowed as structured refuge in the US, but when allowed as a structured refuge in some country or region, seed mixes (refuge provided in the bag) are included in the definition of structured refuge as used herein. Using the current invention, the amount of non-Bt plant seeds in a seed mix targeted at controlling S. frugiperda (e.g., a bag of seed labeled with the fact that can be used to control this insect species) can be lower (compared to when only a single Bt protein-encoding gene is used, or when a Cry1A and a Cry1F protein-encoding gene are combined), provided that Bt-plant seeds contain a Cry1A or Cry1F protein-encoding gene and a VIP3 protein-encoding gene in accordance with this invention.

Further provided herein is a process for growing, sowing or planting seeds or plants expressing a Cry protein or VIP3 protein for control of Spodoptera insects, particularly Spodoptera frugiperda, comprising the step of planting, sowing or growing a structured refuge area of less than 20%, less than 15%, less than 10%, or less than 5%, or an insecticide sprayed structured refuge area of less than 20%, less than 15%, or less than 10% or an non-insecticide sprayed structured refuge area of less than 15%, or less than 10%, or less than 5%, of the planted field or in the vicinity of the planted field, or without planting, sowing or growing a structured refuge area in a field, wherein such structured refuge area is as defined above, particularly in the same field or is within 2 miles, within 1 mile or within 0.5 or 0.25 miles of a field, and which contains plants not comprising such Cry or VIP3 protein, wherein such plants expressing a Cry or VIP3 protein express a combination of a VIP3A protein insecticidal to said insect species, and a Cry1A or Cry1F protein, particularly a VIP3Aa1, VIP3Af1, VIP3Aa19 or VIP3Aa20 and a Cry1Ab, Cry1A.105, Cry1Ac or Cry1F protein, preferably a VIP3Aa and Cry1Ab or Cry1A.105 and Cry1F protein, insecticidal to said insect species. Also provided herein is a field of plants, particularly corn, soybean, rice, sugarcane or cotton plants, comprising a structured refuge of less than 20%, of less than 15%, of less than 10%, or of less than 5%, or comprising no structured refuge (meaning the entire field is planted with the Bt-plants), wherein said field is planted with plants expressing a combination of a VIP3A protein insecticidal to Spodoptera frugiperda insects, and a Cry1A or Cry1F protein, particularly a VIP3Aa1, VIP3Af1, VIP3Aa19 or VIP3Aa20 and a Cry1Ab, Cry1A.105, Cry1Ac or Cry1F protein, preferably a VIP3Aa and Cry1A.105 and Cry1F protein, insecticidal to said insect species.

Further provided herein is a method for deregulating or for obtaining regulatory approval for planting or commercialization of plants expressing proteins insecticidal to Spodoptera frugiperda, or for obtaining a reduction in structured refuge area containing plants not producing any protein insecticidal to such insect species, or for planting fields without a structured refuge area, such method comprising the step of referring to, submitting or relying on insect assay binding data showing that VIP3A proteins bind specifically and saturably to the insect midgut membrane of such insects, and that said VIP3A proteins do not compete with binding sites for Cry1A or Cry1F proteins in such insects, such as the data disclosed herein or similar data reported in another document. In one embodiment such VIP3A protein is a VIP3Aa1, VIP3Af1, VIP3Aa19 or VIP3Aa20 protein and such Cry1A protein is a Cry1Ac, Cry1Ab, or a Cry1Ac or Cry1Ab hybrid protein, such as a Cry1A.105 protein (e.g., the protein of SEQ ID No. 7 or a protein comprising the smallest toxic fragment thereof).

Further provided herein is a field planted with plants containing insecticidal proteins to protect said plants from Spodoptera frugiperda insects, wherein said field has a structured refuge of less than 20%, of less than 10%, or a structured refuge of less than 5%, or has no structured refuge in said field, and wherein said plants express a combination of a) a VIP3A protein insecticidal to said insect species and b) a Cry1A or Cry1F protein insecticidal to said insect species, in said plants. Said plants are preferably corn, rice, sugarcane, soybean or cotton plants.

Also provided herein is a field of plants, particularly corn or cotton plants, comprising a structured refuge of less than 20%, of less than 15%, of less than 10%, or of less than 5%, or comprising no structured refuge, wherein said field is planted with plants expressing a combination of a VIP3Aa or VIP3Af protein insecticidal to S. frugiperda insects, and a Cry1A or Cry1F protein, particularly a VIP3Aa1, VIP3Aa19, VIP3Aa20 or VIP3Af1 protein and a Cry1Ab, Cry1A.105, Cry1Ac or Cry1F protein, preferably a VIP3Aa and Cry1A.105 and Cry1F protein, insecticidal to said insect species.

Also included herein are the above methods, uses or plants, wherein besides the Cry or VIP3 proteins, also a Bt toxin enhancer protein is expressed in said plants, wherein said Bt toxin enhancer protein is a protein or a fragments thereof which is a part, preferably a part comprising or corresponding to the binding domain, of a Bt (Cry or VIP) toxin receptor in an insect, such as a fragment of a cadherin-like protein. These Bt toxin enhancer proteins are fed to target insects together with one or more Bt insecticidal toxins such as Cry proteins, e.g., by expression in the same plants as the Cry or VIP proteins. These Bt toxin enhancer proteins can enhance the toxin activity of the Bt insecticidal protein against the insect species that was the source of the receptor but also against other insect species. In one embodiment, said Bt toxin enhancer protein is a part of a midgut cell Bt toxin receptor of a S. frugiperda insect.

In one embodiment of this invention, the VIP3 and/or Cry1 protein, are expressed at a high dose in the plants used in the invention. ‘High dose’ expression, as used herein when referring to the plants used in the invention, refers to a concentration of the insecticidal protein in a plant (measured by ELISA as a percentage of the total soluble protein, which total soluble protein is measured after extraction of soluble proteins in a standard extraction buffer using Bradford analysis (Bio-Rad, Richmond, Calif.; Bradford, 1976)) which kills at least 95% of insects in a developmental stage of the target insect which is significantly less susceptible, preferably at least 25 times less susceptible to the insecticidal protein than the first larval stage of the insect (as can be analyzed in standard insecticidal protein bio-assays), and can thus can be expected to ensure full control of the target insect species.

General procedures for the evaluation and exploitation of at least two insecticidal genes for prevention of the development, in a target insect, of resistance to transgenic plants expressing those genes can be found in published European patent application EP408403.

In accordance with this invention, the binding of VIP3, Cry1A and Cry1F proteins to the brush border membrane of the midgut cells of Spodoptera frugiperda insect larvae has been investigated. The brush border membrane is the primary target of the VIP or Cry proteins, and membrane vesicles, preferentially derived from the insect midgut brush border membrane, can be obtained according to procedures known in the art, e.g., Wolfersberger et al. (1987).

This invention involves the combined expression of at least two insecticidal protein genes in transgenic plants to delay or prevent resistance development in populations of the target insect Spodoptera frugiperda. The genes are inserted in a plant cell genome, preferably in its nuclear or chloroplast genome, so that the inserted genes are downstream of, and operably linked to, a promoter which can direct the expression of the genes in plant cells.

In one embodiment of this invention is provided a plant with a lasting resistance to Spodoptera frugiperda, said plant comprising a chimeric gene encoding a VIP3 protein insecticidal to Spodoptera frugiperda, and a chimeric gene encoding a Cry1A and/or Cry1F protein, preferably a Cry1F protein or a Cry1A.105 protein as defined above, insecticidal to Spodoptera frugiperda.

In order to express all or an insecticidally effective part of the DNA sequence encoding a VIP3 or Cry1 protein in E. coli, in other Bt strains and in plants, suitable restriction sites can be introduced, flanking the DNA sequence. This can be done by site-directed mutagenesis, using well-known procedures (Stanssens et al., 1989; White et al., 1989). In order to obtain improved expression in plants, the codon usage of the genes or insecticidally effective gene part of this invention can be modified to form an equivalent, modified or artificial gene or gene part in accordance with PCT publications WO 91/16432 and WO 93/09218 and publications EP 0 385 962, EP 0 359 472 and U.S. Pat. No. 5,689,052, or the genes or gene parts can be inserted in the plastid, mitochondrial or chloroplast genome and expressed there using a suitable promoter (e.g., Mc Bride et al., 1995; U.S. Pat. No. 5,693,507, WO 2004/053133).

Because of the degeneracy of the genetic code, some amino acid codons can be replaced by others without changing the amino acid sequence of the protein. Furthermore, some amino acids can be substituted by other equivalent amino acids without significantly changing, preferably without changing, the insecticidal activity of the protein, at least without changing the insecticidal activity of the protein in a negative way. For example conservative amino acid substitutions within the categories basic (e.g. Arg, H is, Lys), acidic (e.g. Asp, Glu), nonpolar (e.g. Ala, Val, Gly, Leu, Ile, Met) or polar (e.g. Ser, Thr, Cys, Asn, Gln) fall within the scope of the invention as long as the insecticidal activity of the protein is not significantly decreased. In addition non-conservative amino acid substitutions fall within the scope of the invention as long as the insecticidal activity of the protein is not significantly decreased. Variants or equivalents of the DNA sequences of the invention include DNA sequences having a different codon usage compared to the native genes of the VIP3, Cry1F or Cry1A proteins used in this invention but which encode a protein with the same insecticidal activity and with substantially the same, preferably the same, amino acid sequence. The DNA sequences can be codon-optimized by adapting the codon usage to that most preferred in plant genes, particularly to genes native to the plant genus or species of interest (Bennetzen & Hall, 1982; Itakura et al., 1977) using available codon usage tables (e.g. more adapted towards expression in cotton, soybean, corn or rice). Codon usage tables for various plant species are published for example by Ikemura (1993) and Nakamura et al. (2000).

For obtaining enhanced expression in monocot plants such as corn, sugarcane or rice, an intron, preferably a monocot intron, can also be added to the chimeric gene. For example the insertion of the intron of the maize Adh1 gene into the 5′ regulatory region has been shown to enhance expression in maize (Callis et. al., 1987). Likewise, the HSP70 intron, as described in U.S. Pat. No. 5,859,347, may be used to enhance expression. The DNA sequence of the insecticidal protein gene or its insecticidal part can be further changed in a translationally neutral manner, to modify possibly inhibiting DNA sequences present in the gene part by means of site-directed intron insertion and/or by introducing changes to the codon usage, e.g., adapting the codon usage to that most preferred by plants, preferably the specific relevant target plant species/genus (Murray et al., 1989), without changing significantly, preferably without changing, the encoded amino acid sequence.

In one embodiment of the invention, fall armyworms (Spodoptera frugiperda) susceptible to a VIP3 and a Cry1F or Cry1A protein are contacted with a combination of these proteins in insect-controlling amounts, preferably insecticidal amounts, e.g., by expressing these proteins in plants targeted by these armyworms or by transforming plants so that these plants and their descendants contain chimeric genes encoding such proteins. In one embodiment target plants for these armyworms are corn, cotton, rice, sugarcane or soybean plants, particularly in Northern, Central and Southern American countries. The term plant, as used herein, encompasses whole plants as well as parts of plants, such as leaves, stems, flowers or seeds.

The insecticidally effective gene, preferably the chimeric gene, encoding an insecticidally effective portion of the VIP3, Cry1F or Cry1A protein, can be stably inserted in a conventional manner into the nuclear genome of a single plant cell, and the so-transformed plant cell can be used in a conventional manner to produce a transformed plant that is insect-resistant. In this regard, a T-DNA vector, containing the insecticidally effective gene, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO 84/02913 and published European Patent application EPO 242 246 and in Gould et al. (1991). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718. Preferred T-DNA vectors each contain a promoter operably linked to the insecticidally effective gene between T-DNA border sequences, or at least located to the left of the right border sequence. Border sequences are described in Gielen et al. (1984). Of course, other types of vectors can be used to transform the plant cell, using procedures such as direct gene transfer (as described, for example in EP 0 223 247), pollen mediated transformation (as described, for example in EP 0 270 356 and WO 85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as the recently described methods for transforming certain lines of corn (e.g., U.S. Pat. No. 6,140,553; Fromm et al., 1990; Gordon-Kamm et al., 1990) and rice (Shimamoto et al., 1989; Datta et al. 1990) and the method for transforming monocots generally (PCT publication WO 92/09696). For cotton transformation, especially preferred is the method described in PCT patent publication WO 00/71733. For rice transformation, reference is made to the methods described in WO92/09696, WO94/00977 and WO95/06722.

The combined expression of a VIP3 and a Cry1F or Cry1A protein is most useful in plants targeted by (or damaged by) the fall armyworm, including corn (field and sweet corn), grasses such as Bermuda grass, turf grass or forage grasses, alfalfa, bean, barley, buckwheat, cotton, clover, oat, potato, sweet potato, turnip, millet, peanut, rice, ryegrass, sorghum, sugarbeet, soybean, sugarcane, tobacco, wheat, apple, grape, orange, papaya, peach, strawberry, spinach, tomato, cabbage, and cucumber; preferably in corn, cotton, rice, soybean, or sugarcane plants. Hence, the combined use of a VIP3 and a Cry1F or Cry1A protein in accordance with the invention, for delaying or preventing resistance development of fall armyworms is preferably in any one of these plants. The term “corn” is used herein to refer to Zea mays. “Cotton” as used herein refers to Gossypium spp., particularly G. hirsutum and G. barbadense. The term “rice” refers to Oryza spp., particularly O. sativa. “Soybean” refers to Glycine spp, particularly G. max. Sugarcane is used herein to refer to plants of the genus Saccharum, a tall perennial grass of the family Poaceae, native to warm temperate to tropical regions that can be used for sugar extraction.

Transformed plants can be used in a conventional plant breeding scheme to produce more transformed plants with the same characteristics or to introduce the insecticidally effective gene part into other varieties of the same or related plant species. Seeds, which are obtained from the transformed plants, contain the insecticidally effective gene as a stable genomic insert. Cells of the transformed plant can be cultured in a conventional manner to produce the insecticidally effective portion of the VIP3 or Cry1 toxin or protein, which can be recovered for use in conventional insecticide compositions against Lepidoptera.

The insecticidally effective gene is inserted in a plant cell genome so that the inserted gene is downstream (i.e., 3′) of, and under the control of, a promoter which can direct the expression of the gene part in the plant cell (a plant-expressible promoter). This is preferably accomplished by inserting the chimeric gene in the plant cell genome, particularly in the nuclear or plastid (e.g., chloroplast) genome.

Plant-expressible promoters that can be used in the invention include but are not limited to: the strong constitutive 35S promoters (the “35S promoters”) of the cauliflower mosaic virus (CaMV) of isolates CM 1841 (Gardner et al., 1981), CabbB-S (Franck et al., 1980) and CabbB-JI (Hull and Howell, 1987); the ³⁵S promoter described by Odell et al. (1985), promoters from the ubiquitin family (e.g., the maize ubiquitin promoter of Christensen et al., 1992, EP 0 342 926, see also Cornejo et al., 1993), the gos2 promoter (de Pater et al., 1992), the emu promoter (Last et al., 1990), Arabidopsis actin promoters such as the promoter described by An et al. (1996), rice actin promoters such as the promoter described by Zhang et al. (1991) and the promoter described in U.S. Pat. No. 5,641,876; promoters of the Cassava vein mosaic virus (WO 97/48819, Verdaguer et al. (1998)), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T-DNA (Velten et al., 1984). Alternatively, a promoter can be utilized which is not constitutive but rather is specific for one or more tissues or organs of the plant (e.g., leaves and/or roots) whereby the inserted gene part is expressed only in cells of the specific tissue(s) or organ(s). For example, the insecticidally effective gene could be selectively expressed in the leaves of a plant (e.g., corn, cotton, rice, soybean) by placing the insecticidally effective gene part under the control of a light-inducible promoter such as the promoter of the ribulose-1,5-bisphosphate carboxylase small subunit gene of the plant itself or of another plant, such as pea, as disclosed in U.S. Pat. No. 5,254,799. The promoter can, for example, be chosen so that the gene of the invention is only expressed in those tissues or cells on which the target insect pest feeds so that feeding by the susceptible target insect will result in reduced insect damage to the host plant, compared to plants which do not express the gene. Another alternative is to use a promoter whose expression is inducible, e.g., the MPI promoter described by Cordera et al. (1994), which is induced by wounding (such as caused by insect feeding), or a promoter inducible by a chemical, such as dexamethasone as described by Aoyama and Chua (1997) or a promoter inducible by temperature, such as the heat shock promoter described in U.S. Pat. No. 5,447,858, or a promoter inducible by other external stimuli.

The insecticidally effective gene is inserted into the plant genome so that the inserted gene is upstream (i.e., 5′) of suitable 3′ end transcription regulation signals (i.e., transcript formation and polyadenylation signals). This is preferably accomplished by inserting the chimeric gene in the plant cell genome. The type of polyadenylation and transcript formation signals is not critical, and can include those of the CaMV 35S gene, the nopaline synthase gene (Depicker et al., 1982), the octopine synthase gene (Gielen et al., 1984) or the T-DNA gene 7 (Velten and Schell, 1985), which act as 3′-untranslated DNA sequences in transformed plant cells.

The selection of marker genes for the chimaeric genes of this invention also is not critical, and any conventional DNA sequence can be used which encodes a protein or polypeptide which renders plant cells, expressing the DNA sequence, readily distinguishable from plant cells not expressing the DNA sequence (EP 0344029). The marker gene can be under the control of its own promoter and have its own 3′ non-translated DNA sequence as disclosed above, provided the marker gene is in the same genetic locus as the gene(s) which it identifies. The marker gene can be, for example: a herbicide resistance gene such as the sfr or sfrv genes (EPA 87400141); a gene encoding a modified target enzyme for a herbicide having a lower affinity for the herbicide than the natural (non-modified) target enzyme, such as a modified 5-EPSP as a target for glyphosate (U.S. Pat. No. 4,535,060; EP 0218571) or a modified glutamine synthetase as a target for a glutamine synthetase inhibitor (EP 0240972); or an antibiotic resistance gene, such as a neo gene (PCT publication WO 84/02913; EP 0193259).

Different conventional procedures can be followed to obtain a combined expression of two insecticidal protein genes in transgenic plants, as summarized in EP 408403, incorporated herein by reference. These include transformation of single genes in different plants and crossing such plants, crossing plants already having incorporated each of the desired genes, retransformation of plant already transformed with one gene with the second gene, cotransformation of plants using different plasmids, transformation with two genes on one transforming DNA so the genes are inserted at the same locus, using translational fusion genes (see, e.g., Ho et al. (2006)) for transformation, and the like.

The transgenic plant obtained can be used in further plant breeding schemes. The transformed plant can be selfed to obtain a plant which is homozygous for the inserted genes. If the plant is an inbred line, this homozygous plant can be used to produce seeds directly or as a parental line for a hybrid variety. The gene can also be crossed into open pollinated populations or other inbred lines of the same plant using conventional plant breeding approaches.

The following Examples illustrate the invention, and are not provided to limit the invention or the protection sought.

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Sequence Listing:

SEQ ID No. 1: Cry1Fa1 protein
SEQ ID No. 2: VIP3Aa1 protein
SEQ ID No. 3: VIP3Af1 protein
SEQ ID No. 4: VIP3Aa19 protein
SEQ ID No. 5: VIP3Aa20 protein
SEQ ID No. 6: Cry1Ac1 protein
SEQ ID No. 7: Cry1A.105 protein
SEQ ID No. 8: Cry1Ab1 protein
SEQ ID No. 9: Cry1F.6275 protein encoded by the cry1 F transgene in corn events TC-6275 and TC1507
SEQ ID No. 10: Cry1F.281-24-236 protein encoded by the transgene in cotton event 281-24-236
SEQ ID No. 11: Cry1Ac.3006-210-23 protein encoded by the transgene in cotton event 3006-210-23

### EXAMPLES

### Example 1

### 1. Materials and Methods

### Preparation of Toxins

The Cry toxins Cry1Ab and Cry1Fa were obtained from recombinant Bt strains expressing a single toxin. The strains were grown for 48 hours in CCY medium (Stewart et al 1981) supplemented with the appropriate antibiotics. Spores and crystals were collected by centrifugation at 9700×g for 10 min at 4° C. The pellet was washed 4 times with 1 M NaCl/10 mM EDTA and was resuspended in 10 mM KCl and solubilized in 50 mM Na₂CO₃ (pH 10.5) including 10 mM DTT. The toxins were activated with trypsin and purified by anion exchange chromatography (Sayyed et al., 2000). The protein concentration was measured using the Bradford method (Bradford, 1976).

### Subcloning of the VIP3Aa1 Gene.

The VIP toxins used in this study were VIP3Af1 (NCBI accession CAI43275) and VIP3Aa1 (NCBI accession AAC37036). The corresponding genes had been cloned in plasmids pNN814 and pGA85, respectively, and were present in E. coli strain WK6. The E. coli strain containing the expression vector pNN814 with the VIP3Af1 gene was suitable for induction and production of the toxin and purification of the toxin by chromatography, since the gene already contained the His tag sequence. In order to express and purify the VIP3Aa1 toxin, it was necessary to subclone the corresponding gene by PCR using plasmid pGA85, a forward primer containing a Ncol site and a His tag sequence (encoding six histidin residues) at the 5′ end, and a reverse primer containing a HamHI site at the 3′ end. The sequence of this gene can be found under GenBank accession number L48811. Following amplification, using Expand High Fidelity Taq polymerase (Roche), and following digestion with Ncol and BamHI and column purification (Kit GFX, Amersham), the fragment was ligated to vector pGEM T-easy (Promega) using the “Rapid DNA Ligation” kit (Roche). Transformation was done in E. coli XL1-Blue competent cells, using the heat shock method (Hanahan, 1983). Recombinant clones were selected on LB medium containing ampicilin (50 μg/ml) and X-Gal. Plasmid DNA from one positive clone was digested with Ncol and BamHI and cloned into the expression vector pNN814 resulting in plasmid pNN814-VIP3Aa1.

Expression and Purification of VIP3 Proteins.

One single colony of E. coli harboring pNN814, with either the VIP3Aa1 gene or the VIP3Af1 gene, was inoculated in a preculture containing 20 ml LB medium containing ampicilin (100 μg/ml) and grown at 37° C. during 16 hours at 250 rpm agitation. The preculture was transferred to 200 ml LB containing ampicilin (100 μg/ml) when the OD600 reached 0.025. When the OD600 reached 1.2, 100 mM IPTG was added for induction. The culture was grown overnight at 37° C. at 190 rpm agitation. Cells were centrifuged using a GSA rotor at 12000 rpm for 30 min. The pellet was resuspended in 20 mM phosphate buffer, pH 7.4, containing 0.5 M NaCl, 100 mg/ml lysozyme, 1 mg/ml DNAse and incubated for 30 min at 37° C. The pellet was then sonicated twice during 60 sec, with a 10 sec pause in between. The supernatant was collected following centrifugation at 14000 rpm. This supernatant was used in bioassays. In order to purify the VIP3 toxins, imidazol was added to a final concentration of 10 mM, and the solution was centrifuged at 14000 rpm for 10 min. The supernatant was loaded on a HiTrap column (Amersham) and eluted with elution buffer (50 mM phosphate buffer pH 8.0 containing 0.3M NaCl and 100 mM imidazol. 1 ml fractions were collected in eppendorf tubes containing 200 μl glycerol. For use in the binding assays, the VIP3 proteins were treated with trypsin using 1% trypsin at 37° C. for 1 hour, and then purified on a MonoQ HR5/5 column (Pharmacia). The protein concentration was determined using the Bradford method.

### Toxin Labeling

The chromatographically purified Cry1Ab toxin was labeled using Na¹²⁵I (Amersham) using the Chloramin-T method (Van Rie et al., 1989). 26 μg toxin was labeled using 0.3 mCi ¹²⁵I. The VIP3 toxins were labeled with biotin using the ECL Protein Biotinylation Module kit (Amersham). The toxins were eluted from the Sephadex G25 column (Amersham) in PBS buffer, pH 7.4. The collected fractions were spotted on nitrocellulose membrane (Hybond C-Super, Amersham) for dot blot analysis. The membranes were incubated with streptavidin-AP conjugate (Roche) and detection was done using NBT-BCIP (Roche). Cry1F was biotinylated using the same procedure.

### Binding of Biotinylated Cry1F, VIP3af1 and VIP3Aa1

Cry1F was incubated for 1 hour with Spodoptera frugiperda BBMV in 100 μl binding buffer (PBS pH 7.5, containing 0.1% BSA). BBMV were washed twice in 500 μl binding buffer and resuspended in 10 μA Milli-Q water and 5 μA sample buffer (Laemli, 1970). The samples were subjected to SDS-PAGE electrophoresis and then blotted onto a nitrocellulose membrane (Hybond ECL, Amersham). The membranes were incubated with streptavidin-AP conjugate (Roche) and detection of biotinylated toxins was done using NBT-BCIP (Roche). 20 μg of BBMV was used with 50 ng of biotinylated Cry1F or 60 ng biotinylated VIP3 protein. In competition assays, at least a 200-fold excess competitor toxin was used.

### Binding of ¹²⁵I-labeled Cry1Ab

The binding experiments were performed as described by Ferré et al. (1991) using appropriate conditions for S. frugiperda with respect to incubation time, BBMV concentration, concentration of labeled toxin and unlabeled toxin. In order to determine an appropriate BBMV concentration to be used, different concentrations of BBMV were used with a fixed concentration of labeled Cry1Ab. The non-specific binding was determined in the presence of a 100 fold excess unlabeled toxin. For competition binding experiments, 7 μg BBMV were incubated with ¹²⁵I labeled Cry1Ab (1.3 nM) in the presence of increasing concentrations of unlabeled toxins (Cry1Ab, Cry1Fa, VIP3Af1 and VIP3Aa1) in a final volume of 0.1 ml binding buffer for 1 hour at ambient temperature. Following incubation, the samples were centrifuged at 16,000×g for 10 min, and the pellets were washed twice with 0.5 ml ice cold binding buffer. Radioactivity in the sample was detected in a Compugamma CS gamma counter (LKB Pharmacia). Experiments were replicated three times and the data were analyzed using the LIGAND program (Munson & Rodbard, 1980) in order to estimate the K_{d} and Rₜ values. The GraphPad Prism version 3.2 program was used to perform t-tests and to construct graphs.

### Bioassays

S. frugiperda larvae were reared on artificial diet as described by Chalfant (1975). Seven different concentrations of activated toxins were tested, and for each concentration 16 neonate larvae were used. A constant volume of 50 μl of the sample dilutions were applied on the artificial diet contained in multiwell plates (Corning). One first instar larvae was placed in each well. The plates were incubated at 25° C. under a relative humidity of 65+/−5% and a photoperiod of 14:10 (light:dark). Mortality was evaluated after 7 days (Aranda et al., 1996). Toxicity data were analyzed using the POLO-PC probit analysis program (from LeOra Software, Berkely, Calif.; see Robertson & Preisler, 1992).

### 2. Results

Binding Assays with Biotinylated Toxins

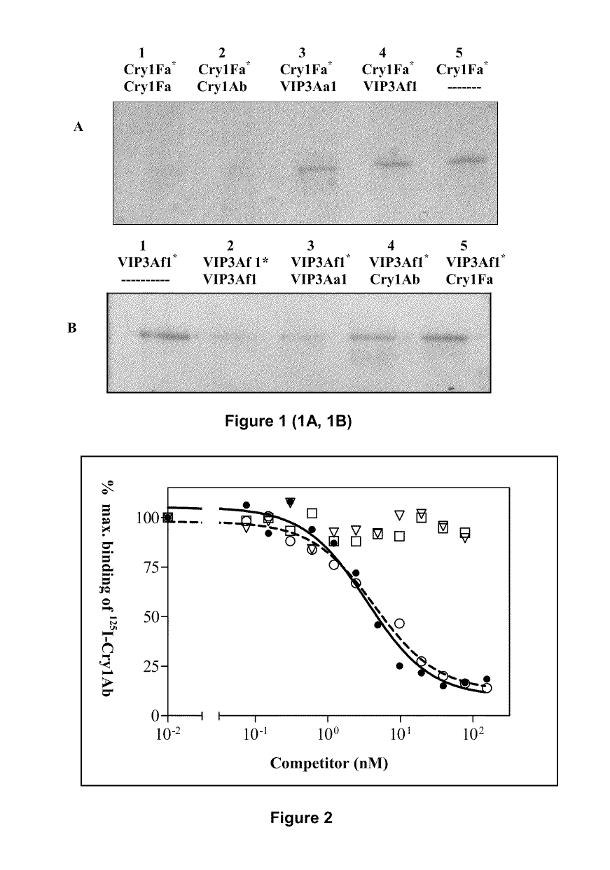

In order to evaluate the binding characteristics of the selected toxins to receptors in S. frugiperda BBMV, and to verify whether these toxins recognize different binding sites, qualitative experiments were performed with the biotinylated VIP3Af and Cry1Fa toxins. These toxins display specific binding to these BBMV (FIG. 1). Indeed, an excess of the same (i.e., homologous) unlabeled toxin reduces significantly the binding of the labeled toxins (compare lanes 5A versus 1A and lanes 1B versus 2B, FIG. 1).

Based on these results it can be concluded that Cry1Fa recognizes the same site as Cry1Ab in S. frugiperda, since the latter toxin significantly reduced the amount of bound labeled Cry1Fa (see lane 2A, FIG. 1). Cry1Fa binding was not reduced by VIP3Aa or VIP3Af toxins (see lanes 3A and 4A), indicating that these toxins recognize another binding site in S. frugiperda midguts. Unlabeled VIP3Aa1 substantially reduces the binding of labeled VIP3Af1, indicating that both toxins recognize the same binding site (see lane 3B). Cry1Ab and Cry1Fa do not compete for this site (see lanes 4B and 5B). These data show that S. frugiperda has a binding site for Cry1Fa, shared with Cry1Ab, and another binding site shared between VIP3Af1 and VIP3Aa1.

FIG. 1 (enclosed below) shows the binding of biotinylated toxins Cry1Fa (A), VIP3Af1 (B) to S. frugiperda BBMV, in absence of competitor (lanes A5, B1) or in the presence of a 200 fold excess of competitor (Cry1Fa, Cry1Ab, VIP3Af1, and VIP3Aa1). The biotinylated toxins were incubated with BBMV and were subjected to SDS-PAGE analysis. Following transfer to nitrocellulose membranes, the labeled toxins were detected using BCIP-NBT. These experiments were repeated 2 to 3 times.

Similar results as in FIG. 1B are obtained when using a labelled VIP3Aa toxin and the same competitor molecules as in FIG. 1B.

Binding Assays with Radiolabeled Cry1Ab to S. frugiperda BBMV

Preliminary experiments were performed in order to determine whether Cry1Ab binds specifically to S. frugiperda BBMV and to identify an appropriate BBMV concentration to perform competition binding experiments. ¹²⁵I-labeled Cry1Ab was incubated with various concentrations of BBMV. The maximum binding of Cry1Ab was observed at concentrations of 0.05 to 0.15 mg BBMV/ml.

Homologous competition experiments were performed using ¹²⁵I-labeled Cry1Ab and increasing concentrations of unlabeled Cry1Ab (FIG. 2). It can be observed that labeled Cry1Ab is almost completely displaced by unlabeled Cry1Ab.

Heterologous competition experiments, using unlabeled Cry1Fa, VIP3Af1 and VIP3Aa1, were performed in order to assess whether the Cry1Ab binding site is recognized by the other toxins. FIG. 2 shows that labeled Cry1Ab was displaced by Cry1Fa, indicating that Cry1Fa recognizes all Cry1Ab sites in S. frugiperda. In contrast, labeled Cry1Ab was not displaced by any of the tested VIP3A toxins. These results demonstrate that there is one high affinity site for the studied Cry1 toxins (Cry1F and Cry1Ab) and another site for the studied VIP3A toxins (VIP3Aa and VIP3Af). These results are in agreement with those obtained using the biotinylated toxins.

FIG. 2 (included below) shows the competition between ¹²⁵I labeled Cry1Ab and unlabeled toxins (Cry1Ab (, filled circle), Cry1Fa (∘, empty circle), VIP3Aa1 (▭, empty rectangle) and VIP3Af1 (∇, empty triangle upside down)). S. frugiperda BBMV were incubated with ¹²⁵I labeled Cry1Ab and different concentrations of unlabeled toxins. Binding was expressed as a percentage of the maximum level of binding of labeled toxin in the absence of unlabeled toxin. Each data point is the average based on results from two independent experiments.

### Bioassays

The potency of the Cry1Ab, Cry1Fa, VIP3Af1 and VIP3Aa1 toxins for S. frugiperda was tested using neonate larvae. The Cry toxins were used as trypsin-treated toxins, whereas the VIP3A toxins were tested without protease treatment. The results, summarized in Table 1 below, show that VIP3Aa1 and VIP3Af1 were highly toxic to S. frugiperda (LC50 values of 49.3 and 21.0 ng/cm², respectively). Cry1Fa also exhibited toxicity to S. frugiperda, corroborating data found by Luo et al. (1999), who found a value of 109 (31-168) ng/cm². Cry1Ab had the weakest activity (LC50: 866.6 ng/cm²).

Interestingly, it is found that the VIP3Af protein is about twice more active to S. frugiperda larvae compared to the VIP3Aa protein.

TABLE 1

| Toxins | LC₅₀ (ng/cm²) (CL min-max)¹ |
|---|---|
| VIP3Aa1 | 49.3 (32.6-71.4) |
| VIP3Af1 | 21.0 (13.0-31.7) |
| Cry1Ab | 867 (539-1215) |
| Cry1Fa | 170 (128-224) |

¹95% confidence level

### Example 2

Several procedures can be envisaged for obtaining the combined expression of two insecticidal protein genes, such as the VIP3A and cry1F or cry1Ab genes in transgenic plants, such as corn or cotton plants.

A first procedure is based on sequential transformation steps in which a plant, already transformed with a first chimeric gene, is retransformed in order to introduce a second gene. The sequential transformation preferably makes use of two different selectable marker genes, such as the resistance genes for kanamycin and phosphinotricin acetyl transferase (e.g., the well known pat or bar genes), which confers resistance to glufosinate herbicides. The use of both these selectable markers has been described in De Block et al. (1987).

The second procedure is based on the cotransformation of two chimeric genes encoding different insecticidal proteins on different plasmids in a single step. The integration of both genes can be selected by making use of the selectable markers, linked with the respective genes.

Also, separate transfer of two insecticidal protein genes to the nuclear genome of separate plants can be done in independent transformation events, which can subsequently be combined in a single plant through crossing. E.g., corn plants comprising the MIR162 event (WO 2007/142840, USDA APHIS petition for non-regulated status 07-253-01p) are crossed with corn plants containing event TC1507 (USDA APHIS petition for non-regulated status 00-136-01p), creating corn plants expressing a VIP3A and a Cry1F insect control protein. Alternatively, corn plants comprising the MIR162 event (WO 2007/142840, USDA APHIS petition for non-regulated status 07-253-01p) are crossed with corn plants containing event Bt11 (USDA APHIS petition for non-regulated status 95-195-01p) or corn plants containing event MON810 (USDA APHIS petition 96-017-01p), creating corn plants expressing a VIP3A and a Cry1Ab insect control protein

Parts of these stacked corn plants can be provided as feed to Spodoptera frugiperda insects, and can be compared to transgenic corn plants expressing only a Cry1F or a Cry1Ab protein, or plants expressing a Cry1F and Cry1Ab protein (such as a cross of TC1507 corn with MON810 or Bt11 corn). When several generations of insects of S. frugiperda (freshly collected from the field) are fed on this plant material at a suitable dose in the lab (e.g., by providing a mixture of non-Bt and Bt corn plant material, ideally blended), the resistance development of this S. frugiperda population to corn plants expressing the two insect control proteins VIP3Aa and Cry1F or VIP3Aa and Cry1Ab can be compared to the resistance development to corn plants expressing only the single proteins, or plants comprising the Cry1Ab and Cry1F proteins.

According to this invention, also cotton plants comprising the event 281-24-236 (as defined in the description, or alternatively, any Widestrike™ cotton line containing this event) can be crossed with the COT102 cotton event (as defined in the description), so that both the Cry1F (and Cry1A in the case of a Widestrike™ cotton line) and the VIP3A proteins are expressed in the same cotton plants.

Co-expression of the two insecticidal protein genes in the individual transformants can be evaluated by insect toxicity tests and by biochemical means known in the art. Specific probes allow for the quantitive analysis of the transcript levels; monoclonal antibodies cross-reacting with the respective gene products allow the quantitative analysis of the respective gene products in ELISA tests; and specific DNA probes allow the characterization of the genomic integrations of the transgenes in the transformants.

Of course, besides the above combinations of VIP3 and Cry1 genes for insect resistance management towards fall armyworms, these plants can also comprise other transgenes, such as genes conferring protection to other Lepidopteran insect species or to insect species from other insect orders, such as Coleopteran or Homopteran insect species, or genes conferring tolerance to herbicides, and the like.

All patents, patent applications, and publications or public disclosures (including publications on internet, and petitions for non-regulated status) referred to or cited herein are incorporated by reference in their entirety to the extent they are not inconsistent with the explicit teachings of this specification. The citation of any document herein does not mean that such document forms part of the common general knowledge in the art.

### CITED REFERENCES

An et al. (1996) Plant J. 10, 107
Aranda et al. (1996) J. Invertebr. Pathol. 68, 203-212
Aoyama and Chua (1997) Plant Journal 11, 605-612
Ballester et al. (1999) Appl. Environm. Microbiol. 65, 1413-1419
Bennetzen & Hall (1982) J. Biol. Chem. 257, 3026-3031
Bradford (1976) Anal Biochem 72, 248-254
Callis et. al. (1987) Genes Developm. 1, 1183-1200
Chalfant (1975) J. Georgia Entomol. Soc. 10, 32-33
Christensen et al. (1992) Plant Mol. Biol. 18, 675-689
Cordera et al. (1994) The Plant Journal 6, 141
Cornejo et al. (1993) Plant Mol. Biol. 23, 567-581
Crickmore et al. (2008) “Bacillus thuringiensis toxin nomenclature”. www.lifesci.sussex.ac.uk/Home/Neil_Crickmore/Bt/
Datta et al. (1990) Bio/Technology 8, 736-740
De Block et al. (1987) EMBO J. 6, 2513-2518
De Pater et al. (1992) Plant J. 2, 834-844
Depicker et al. (1982) J. Molec. Appl. Genetics 1, 561-573
EPA experimental use permit factsheet 006499 (2007) Bacillus thuringiensis Vip3Aa19 protein and the genetic material necessary for its production (vector pCOT1) in Event COT102 cotton plants (006499) Experimental Use Permit Factsheet (Environmental Protection Agency, USA, www.epa.gov)
Estruch et al. (1996) Proc Natl Acad Sci USA. 93, 5389-94
Fang et al. (2007) Appl. Environm. Microbiol. 73, 956-961
Ferré et al. (1991) Proc. Natl. Acad. Sci. USA 88, 5119-5123
Fiuza et al. (1996) Appl. Environm. Microbiol. 62, 1544-1549
Franck et al. (1980) Cell 21, 285-294
Fromm et al. (1990) Bio/Technology 8, 833-839
Gardner et al. (1981) Nucleic Acids Research 9, 2871-2887
Gielen et al. (1984) EMBO J. 3, 835-845
Gordon-Kamm et al. (1990) The Plant Cell 2, 603-618
Gould et al. (1991) Plant Physiol. 95, 426-434
Hanahan (1983) J. Mol. Biol. 166, 557-580
Henikoff and Henikoff (1992) Proc. Natl. Academy Science 89, 915-919
Ho et al. (2006) Crop Sci 46, 781-789
Hofmann et al. (1988), Proc. Natl. Acad. Sci. U.S.A. 85, 7844-7848
Hull and Howell (1987) Virology 86, 482-493
Ikemura, 1993, In “Plant Molecular Biology Labfax”, Croy, ed., Bios Scientific Publishers Ltd.
Itakura et al.(1977). Science 198, 1056-1063
Laemli et al. (1970) Nature 227, 680-685
Last et al. (1990) Theor. Appl. Genet. 81, 581-588
Lee et al. (2003) Appl. Environm. Microbiol. 69, 4648-4657
Luo et al. (1999) Appl. Environm. Microbiol. 65, 457-464
McBride et al.(1995) Bio/Technology 13, 362
Munson & Rodbard (1980) Anal. Biochem. 107, 220-239
Murray et al. (1989) Nucleic Acids Research 17, 477-498
Nakamura et al. (2000) Nucl. Acids Res. 28, 292
Needleman and Wunsch algorithm (1970) J. Mol. Biol. 48, 443-453
Nielsen et al. (1996) Int. J. Neur. Sys., 8, 581-599, 1997
Odell et al. (1985) Nature 313, 810-812
Rang et al. (2004) Curr. Microbiol. 49, 22-27
Robertson & Preisler (1992) CRC Press, Boca Raton, Fla., USA, 127 p
Sayyed et al. (2000). Appl. Environm. Microb. 66, 1509-1516
Shimamoto et al (1989) Nature 338, 274-276
Stanssens et al.(1989) Nucleic Acids Research 12, 4441-4454
Stewart et al (1981) Biochem. J. 198, 101-106
USDA-APHIS petition for non-regulated status 06-298-01p (2006) for corn event MON 89034, see notice published in the US Federal Register on Dec. 13, 2007 (72 FR 70817-70819; Docket No. APHIS-2007-0030).
Van Rie et al. (1989) Eur. J. Biochem. 186, 239-247
Velten et al. (1984) EMBO J. 3, 2723-2730
Velten and Schell (1985), Nucleic Acids Research 13, 6981-6998
Verdaguer et al. (1998), Plant Mol. Biol. 37, 1055-1067
Von Heijne, Gunnar (1986) Nucl. Acids Res. 14:11, 4683-4690
Wolfersberger et al. (1987), Comp. Biochem. Physiol. 86, 301-308
White et al.(1989). Trends in Genet. 5, 185-189
Zhang et al. (1991) The Plant Cell 3, 1155-1165

TABLE 2
VIP3 protein list (www.lifesci.susx.ac.uk/home/Neil_Crickmore/Bt/vip.html)

| | | NCBI | | |
|---|---|---|---|---|
| VIP3 | Prior name | accession | author | Other reference |
| Vip3Aa1 | Vip3Aa | AAC37036 | Estruch et al | PNAS 93, 5389-5394 |
| Vip3Aa2 | Vip3Ab | AAC37037 | Estruch et al | PNAS 93, 5389-5394 |
| Vip3Aa3 | Vip3Ac | | Estruch et al | U.S. Pat. No. 6,137,033 |
| Vip3Aa4 | PS36A Sup | AAR81079 | Feitelson et al | U.S. Pat. No. 6,656,908 |
| Vip3Aa5 | PS81F Sup | AAR81080 | Feitelson et al | U.S. Pat. No. 6,656,908 |
| Vip3Aa6 | Jav90 Sup | AAR81081 | Feitelson et al | U.S. Pat. No. 6,656,908 |
| Vip3Aa7 | Vip83 | AAK95326 | Cai et al | |
| Vip3Aa8 | Vip3A | AAK97481 | Loguercio et al | |
| Vip3Aa9 | VipS | CAA76665 | Selvapandiyan | |
| | | | et al | |
| Vip3Aa10 | Vip3V | AAN60738 | Doss et al | Protein Expr. Purif. 26, 82-88 |
| Vip3Aa11 | Vip3A | AAR36859 | Liu et al | |
| Vip3Aa12 | Vip3A-WB5 | AAM22456 | Wu and Guan | |
| Vip3Aa13 | Vip3A | AAL69542 | Chen et al | Sheng Wu Gong Cheng Xue Bao 18, 687-692 |
| Vip3Aa14 | Vip | AAQ12340 | Polumetla et al | |
| Vip3Aa15 | Vip3A | AAP51131 | Wu et al | |
| Vip3Aa16 | Vip3LB | AAW65132 | Mesrati et al | FEMS Micro Lett 244, 353-358 |
| Vip3Aa17 | Jav90 | | Feitelson et al | U.S. Pat. No. 6,603,063 |
| Vip3Aa18 | | AAX49395 | Cai and Xiao | |
| Vip3Aa19-2 | Vip3ALD | ABB72459 | Liu et al | |
| Vip3Aa19 | Vip3A-1 | ABG20428 | Syngenta | |
| Vip3Aa20 | Vip3A-2 | ABG20429 | Syngenta | |
| Vip3Aa21 | Vip | ABD84410 | Panbangred | |
| Vip3Aa22 | Vip3A-LS1 | AAY41427 | Lu et al | |
| Vip3Aa23 | Vip3A-LS8 | AAY41428 | Lu et al | |
| Vip3Ab1 | Vip3B | AAR40284 | Feitelson et al | U.S. Pat. No. 6,603,063 |
| Vip3Ab2 | Vip3D | AAY88247 | Feng and Shen | |
| Vip3Ac1 | PS49C | | Narva et al | US 20040128716 |
| Vip3Ad1 | PS158C2 | | Narva et al | US 20040128716 |
| Vip3Ad2 | ISP3B | CAI43276 | Van Rie et al | WO 03/080656 |
| Vip3Ae1 | ISP3C | CAI43277 | Van Rie et al | WO 03/080656 |
| Vip3Af1 | ISP3A | CAI43275 | Van Rie et al | WO 03/080656 |
| Vip3Af2 | Vip3C | | Syngenta | WO 03/075655 |
| Vip3Ag1 | Vip3B | | Syngenta | WO 02/078437 |
| Vip3Ah1 | Vip3S | ABH10614 | Li and Shen | |
| Vip3Ba1 | | AAV70653 | Rang et al | |
| Vip3Bb1 | Vip3Z | | Syngenta | WO 03/075655 |

1. A method of controlling Spodoptera frugipera infestation in transgenic plants while securing a slower buildup of Spodoptera frugiperda insect resistance development to said plants, comprising expressing a combination of a) a VIP3 protein insecticidal to said insect species and b) a Cry1A or Cry1F protein insecticidal to said insect species, in said plants.

2. A method for preventing or delaying insect resistance development in populations of the insect species Spodoptera frugiperda to transgenic plants expressing insecticidal proteins to control said insect pest, comprising expressing a VIP3 protein insecticidal to Spodoptera frugiperda in combination with a Cry1F protein insecticidal to Spodoptera frugiperda in said plants.

3. A method to control Spodoptera frugiperda in a region where populations of said insect have become resistant to plants comprising a Cry1F and/or a Cry1A protein, comprising the step of sowing or planting in said region, plants comprising a VIP3 protein insecticidal to Spodoptera frugiperda.

4. A method to control Spodoptera frugiperda in a region where populations of said insect have become resistant to plants comprising a VIP3 protein, comprising the step of sowing or planting in said region, plants comprising a Cry1F and/or Cry1A protein insecticidal to Spodoptera frugiperda.

5. A method for obtaining plants comprising two different insecticidal proteins, wherein said proteins do not share binding sites in larvae of the species Spodoptera frugiperda as determined in competition binding experiments using brush border membrane vesicles of said insect larvae, comprising the step of obtaining plants comprising a plant-expressible chimeric gene encoding a VIP3 protein insecticidal to Spodoptera frugiperda and a plant-expressible chimeric gene encoding a Cry1A or Cry1F protein insecticidal to Spodoptera frugiperda.

6. The method of claim 5, wherein said plants are obtained by transformation of a plant with chimeric genes encoding said VIP3 and said Cry1A or Cry1F proteins, and by obtaining progeny plants and seeds of said plant comprising said chimeric genes.

7. A method of sowing, planting, or growing plants protected against fall armyworms, comprising chimeric genes expressing two different insecticidal proteins, wherein said proteins do not share binding sites in larvae of the species Spodoptera frugiperda as determined in competition binding experiments using brush border membrane vesicles of said larvae, comprising the step of: sowing, planting, or growing plants comprising a chimeric gene encoding a VIP3 protein insecticidal to Spodoptera frugiperda and a chimeric gene encoding a Cry1A or Cry1F protein insecticidal to Spodoptera frugiperda.

8. The method of any one of claims 3 to 7, wherein said plants comprising a VIP3A gene are selected from the group consisting of: corn plants comprising the MIR162 event of USDA APHIS petition 07-253-01p (WO 2007/142840), cotton plants comprising the COT102 event of USDA APHIS petition 03-155-01p (WO 2004/039986), cotton plants comprising the COT202 event described in WO 2005/054479, and cotton plants comprising the COT203 event described in WO 2005/054480.

9. The method of any one of claims 3 to 8, wherein said plants comprising a Cry1A gene are selected from the group consisting of: corn plants comprising the MON810 event of USDA APHIS petition 96-017-01p (U.S. Pat. No. 6,713,259), corn plants comprising the Bt11 event of USDA APHIS petition 95-195-01p (U.S. Pat. No. 6,114,608), cotton plants comprising the COT67B event of USDA APHIS petition 07-108-01p, cotton plants comprising the 3006-210-23 event of USDA APHIS petition 03-036-02p (WO 2005/103266), cotton plants comprising event 531 of USDA APHIS petition 94-308-01p (the Cry1A gene event of WO 2002/100163), and corn plants comprising the MON89034 event of USDA APHIS petition 06-298-01p (the Cry1A gene-containing event of WO 2007/140256).

10. The method of any one of claims 3 to 9, wherein said plants comprising a Cry1F gene are selected from the group consisting of: corn plants comprising the TC1507 event of USDA APHIS petition 00-136-01p (WO 004/099447), corn plants comprising the TC-2675 event of USDA APHIS petition 03-181-01p, cotton plants comprising the 281-24-236 event of USDA APHIS petition 03-036-01p (the Cry1F gene-containing event of WO 2005/103266).

11. The method of any one of claims 3 to 7, wherein said VIP3, Cry1A or Cry1F chimeric genes comprise the VIP3A, Cry1A or Cry1F coding regions selected from any one of the VIP3A, Cry1A or Cry1F coding regions contained in any one of said cotton or corn events of claims 8 to 10, or wherein said VIP3, Cry1A or Cry1F chimeric genes are any one of the VIP3, Cry1F or Cry1A chimeric genes contained in any one of said cotton or corn events.

12. The method of any one of claims 1 to 11, wherein said plant is selected from the group consisting of: cotton, corn, rice, soybean, or sugarcane.

13. The method of any one of claims 1 to 12, wherein said process also includes the planting of a refuge area with plants not comprising a chimeric gene encoding a Cry1or VIP3 protein insecticidal to Spodoptera frugiperda.

14. The method of any one of claims 1 to 13, wherein said plants provide a high dose of Cry1 or VIP3 protein for S. frugiperda.

15. The method of any one of claims 1 to 14, wherein said Cry1A protein is a Cry1Ac, Cry1Ab or Cry1A.105 protein.

16. The method of any one of claims 1 to 15, wherein said VIP3 protein is selected from the group consisting of: a protein insecticidal to S. frugiperda with at least 70% sequence identity with the VIP3Aa1 protein.

17. The method of any one of claims 1 to 16, wherein said VIP3 protein is a VIP3Aa19 protein or a VIP3Aa20 protein.

18-21. (canceled)

22. A method for obtaining a reduction in structured refuge area containing plants not producing any Bt protein insecticidal to S. frugiperda in a field, such method comprising the step of referring to, submitting or relying on insect assay binding data showing that VIP3A proteins do not compete with binding sites for Cry1A or Cry1F proteins in such insect species.

23. (canceled)

24. A field of insect-resistant transgenic plants controlling S. frugiperda insects, wherein said field has a structured refuge area of less than 20%, of less than 15%, of less than 10%, or of less than 5%, or has no structured refuge area, wherein said plants express a combination of a VIP3Aa or VIP3Af protein insecticidal to S. frugiperda insects, and a Cry1A or Cry1F protein insecticidal to S. frugiperda insects, particularly a VIP3Aa1, VIP3Af1, VIP3Aa19 or VIP3Aa20 protein and a Cry1Ab, Cry1A.105, Cry1Ac or Cry1Fa protein insecticidal to S. frugiperda insects, preferably a VIP3Aa, a Cry1A.105 and a Cry1F protein, insecticidal to S. frugiperda insects.


SEQUENCE LISTING (11 sequences)

SEQ ID NO: 1 - protein, 1174 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIREAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 2 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 3 - protein, 788 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGFSQFIGDKLKPKTEYVIQYTVKGKPSIHLKDENTGYIHYEDTNNNLKDYQTITKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSREVLFERSNLMSSTSHISGTFKTESNNTGLYVELSRRSGGGGHISFENVSIK

SEQ ID NO: 4 - protein, 789 aa, Artificial; identical to SEQ ID NO: 2
  designed sequence

SEQ ID NO: 5 - protein, 789 aa, Artificial
  designed sequence
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 6 - protein, 1178 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 7 - protein, 1177 aa, Artificial
  designed sequence
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 8 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 9 - protein, 605 aa, Artificial
  designed sequence
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLE

SEQ ID NO: 10 - protein, 1148 aa, artificial
  designed sequence
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 11 - protein, 1156 aa, artificial
  designed sequence
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE